SEQUENCE LISTING (20 sequences)

SEQ ID NO: 1 - protein, 321 aa, Escherichia coli
MSEALLNAGRQTLMLELQEASRLPERLGDDFVRAANIILHCEGKVVVSGIGKSGHIGKKIAATLASTGTPAFFVHPAEALHGDLGMIESRDVMLFISYSGGAKELDLIIPRLEDKSIALLAMTGKPTSPLGLAAKAVLDISVEREACPMHLAPTSSTVNTLMMGDALAMAVMQARGFNEEDFARSHPAGALGARLLNKVHHLMRRDDAIPQVALTASVMDAMLELSRTGLGLVAVCDAQQQVQGVFTDGDLRRWLVGGGALTTPVNEAMTVGGTTLQSQSRAIDAKEILMKRKITAAPVVDENGKLTGAINLQDFYQAGII

SEQ ID NO: 2 - protein, 328 aa, Escherichia coli
MSHVELQPGFDFQQAGKEVLAIERECLAELDQYINQNFTLACEKMFWCKGKVVVMGMGKSGHIGRKMAATFASTGTPSFFVHPGEAAHGDLGMVTPQDVVIAISNSGESSEITALIPVLKRLHVPLICITGRPESSMARAADVHLCVKVAKEACPLGLAPTSSTTATLVMGDALAVALLKARGFTAEDFALSHPGGALGRKLLLRVNDIMHTGDEIPHVKKTASLRDALLEVTRKNLGMTVICDDNMMIEGIFTDGDLRRVFDMGVDVRQLSIADVMTPGGIRVRPGILAVEALNLMQSRHITSVMVADGDHLLGVLHMHDLLRAGVV

SEQ ID NO: 3 - protein, 284 aa, Escherichia coli
MKQKVVSIGDINVANDLPFVLFGGMNVLESRDLAMRICEHYVTVTQKLGIPYVFKASFDKANRSSIHSYRGPGLEEGMKIFQELKQTFGVKIITDVHEPSQAQPVADVVDVIQLPAFLARQTDLVEAMAKTGAVINVKKPQFVSPGQMGNIVDKFKEGGNEKVILCDRGANFGYDNLVVDMLGFSIMKKVSGNSPVIFDVTHALQCRDPFGAASGGRRAQVAELARAGMAVGLAGLFIEAHPDPEHAKCDGPSALPLAKLEPFLKQMKAIDDLVKGFEELDTSK

SEQ ID NO: 4 - protein, 248 aa, Escherichia coli
MSFVVIIPARYASTRLPGKPLVDINGKPMIVHVLERARESGAERIIVATDHEDVARAVEAAGGEVCMTRADHQSGTERLAEVVEKCAFSDDTVIVNVQGDEPMIPATIIRQVADNLAQRQVGMATLAVPIHNAEEAFNPNAVKVVLDAEGYALYFSRATIPWDRDRFAEGLETVGDNFLRHLGIYGYRAGFIRRYVNWQPSPLEHIEMLEQLRVLWYGEKIHVAVAQEVPGTGVDTPEDLERVRAEMR

SEQ ID NO: 5 - protein, 188 aa, Escherichia coli
MSKAGASLATCYGPVSADVIAKAENIRLLILDVDGVLSDGLIYMGNNGEELKAFNVRDGYGIRCALTSDIEVAIITGRKAKLVEDRCATLGITHLYQGQSNKLIAFSDLLEKLAIAPENVAYVGDDLIDWPVMEKVGLSVAVADAHPLLIPRADYVTRIAGGRGAVREVCDLLLLAQGKLDEAKGQSI

SEQ ID NO: 6 - protein, 425 aa, Escherichia coli
MLELLYTALLYLIQPLIWIRLWVRGRKAPAYRKRWGERYGFYRHPLKPGGIMLHSVSVGETLAAIPLVRALRHRYPDLPITVTTMTPTGSERVQSAFGKDVQHVYLPYDLPDALNRFLNKVDPKLVLIMETELWPNLIAALHKRKIPLVIANARLSARSAAGYAKLGKFVRRLLRRITLIAAQNEEDGARFVALGAKNNQVTVTGSLKFDISVTPQLAAKAVTLRRQWAPHRPVWIATSTHEGEESVVIAAHQALLQQFPNLLLILVPRHPERFPDAINLVRQAGLSYITRSSGEVPSTSTQVVVGDTMGELMLLYGIADLAFVGGSLVERGGHNPLEAAAHAIPVLMGPHTFNFKDICARLEQASGLITVTDATTLAKEVSSLLTDADYRSFYGRHAVEVLYQNQGALQRLLQLLEPYLPPKTH

SEQ ID NO: 7 - protein, 807 aa, Escherichia coli
MYSSSRKRCPKTKWALKLLTAAFLAASPAAKSAVNNAYDALIIEARKGNTQPALSWFALKSALSNNQIADWLQIALWAGQDKQVITVYNRYRHQQLPARGYAAVAVAYRNLQQWQNSLTLWQKALSLEPQNKDYQRGQILTLADAGHYDTALVKLKQLNSGAPDKANLLAEAYIYKLAGRHQDELRAMTESLPENASTQQYPTEYVQALRNNQLAAAIDDANLTPDIRADIHAELVRLSFMPTRSESERYAIADRALAQYAALEILWHDNPDRTAQYQRIQVDHLGALLTRDRYKDVISHYQRLKKTGQIIPPWGQYWVASAYLKDHQPKKAQSIMTELFYHKETIAPDLSDEELADLFYSHLESENYPGALTVTQHTINTSPPFLRLMGTPTSIPNDTWLQGHSFLSTVAKYSNDLPQAEMTARELAYNAPGNQGLRIDYASVLQARGWPRAAENELKKAEVIEPRNINLEVEQAWTALTLQEWQQAAVLTHDVVEREPQDPGVVRLKRAVDVHNLAELRIAGSTGIDAEGPDSGKHDVDLTTIVYSPPLKDNWRGFAGFGYADGQFSEGKGIVRDWLAGVEWRSRNIWLEAEYAERVFNHEHKPGARLSGWYDFNDNWRIGSQLERLSHRVPLRAMKNGVTGNSAQAYVRWYQNERRKYGVSWAFTDFSDSNQRHEVSLEGQERIWSSPYLIVDFLPSLYYEQNTEHDTPYYNPIKTFDIVPAFEASHLLWRSYENSWEQIFSAGVGASWQKHYGTDVVTQLGYGQRISWNDVIDAGATLRWEKRPYDGDREHNLYVEFDMTFRF

SEQ ID NO: 8 - protein, 672 aa, Escherichia coli
MLRNGNKYLLMLVSIIMLTACISQSRTSFIPPQDRESLLAEQPWPHNGFVAISWHNVEDEAADQRFMSVRTSALREQFAWLRENGYQPVSIAQIREAHRGGKPLPEKAVVLTFDDGYQSFYTRVFPILQAFQWPAVWAPVGSWVDTPADKQVKFGDELVDREYFATWQQVREVARSRLVELASHTWNSHYGIQANATGSLLPVYVNRAYFTDHARYETAAEYRERIRLDAVKMTEYLRTKVEVNPHVFVWPYGEANGIAIEELKKLGYDMFFTLESGLANASQLDSIPRVLIANNPSLKEFAQQIITVQEKSPQRIMHIDLDYVYDENLQQMDRNIDVLIQRVKDMQISTVYLQAFADPDGDGLVKEVWFPNRLLPMKADIFSRVAWQLRTRSGVNIYAWMPVLSWDLDPTLTRVKYLPTGEKKAQIHPEQYHRLSPFDDRVRAQVGMLYEDLAGHAAFDGILFHDDALLSDYEDASAPAITAYQQAGFSGSLSEIRQNPEQFKQWARFKSRALTDFTLELSARVKAIRGPHIKTARNIFALPVIQPESEAWFAQNYADFLKSYDWTAIMAMPYLEGVAEKSADQWLIQLTNQIKNIPQAKDKSILELQAQNWQKNGQHQAISSQQLAHWMSLLQLNGVKNYGYYPDNFLHNQPEIDLIRPEFSTAWYPKND

SEQ ID NO: 9 - protein, 441 aa, Escherichia coli
MINRIVSFFILCLVLCIPLCVAYFHSGELMMRFVFFWPFFMSIMWIVGGVYFWVYRERHWPWGENAPAPQLKDNPSISIIIPCFNEEKNVEETIHAALAQRYENIEVIAVNDGSTDKTRAILDRMAAQIPHLRVIHLAQNQGKAIALKTGAAAAKSEYLVCIDGDALLDRDAAAYIVEPMLYNPRVGAVTGNPRIRTRSTLVGKIQVGEYSSIIGLIKRTQRIYGNVFTVSGVIAAFRRSALAEVGYWSDDMITEDIDISWKLQLNQWTIFYEPRALCWILMPETLKGLWKQRLRWAQGGAEVFLKNMTRLWRKENFRMWPLFFEYCLTTIWAFTCLVGFIIYAVQLAGVPLNIELTHIAATHTAGILLCTLCLLQFIVSLMIENRYEHNLTSSLFWIIWFPVIFWMLSLATTLVSFTRVMLMPKKQRARWVSPDRGILRG

SEQ ID NO: 10 - protein, 137 aa, Escherichia coli
MNNLIITTRQSPVRLLVDYVATTILWTLFALFIFLFAMDLLTGYYWQSEARSRLQFYFLLAVANAVVLIVWALYNKLRFQKQQHHAAYQYTPQEYAESLAIPDELYQQLQKSHRMSVHFTSQGQIKMVVSEKALVRA

SEQ ID NO: 11 - protein, 306 aa, Escherichia coli
MTNLPKFSTALLHPRYWLTWLGIGVLWLVVQLPYPVIYRLGCGLGKLALRFMKRRAKIVHRNLELCFPEMSEQERRKMVVKNFESVGMGLMETGMAWFWPDRRIARWTEVIGMEHIRDVQAQKRGILLVGIHFLTLELGARQFGMQEPGIGVYRPNDNPLIDWLQTWGRLRSNKSMLDRKDLKGMIKALKKGEVVWYAPDHDYGPRSSVFVPLFAVEQAATTTGTWMLARMSGACLVPFVPRRKPDGKGYQLIMLPPECSPPLDDAETTAAWMNKVVEKCIMMAPEQYMWLHRRFKTRPEGVPSRY

SEQ ID NO: 12 - protein, 323 aa, Escherichia coli
METKKNNSEYIPEFDKSFRHPRYWGAWLGVAAMAGIALTPPKFRDPILARLGRFAGRLGKSSRRRALINLSLCFPERSEAEREAIVDEMFATAPQAMAMMAELAIRGPEKIQPRVDWQGLEIIEEMRRNNEKVIFLVPHGWAVDIPAMLMASQGQKMAAMFHNQGNPVFDYVWNTVRRRFGGRLHARNDGIKPFIQSVRQGYWGYYLPDQDHGPEHSEFVDFFATYKATLPAIGRLMKVCRARVVPLFPIYDGKTHRLTIQVRPPMDDLLEADDHTIARRMNEEVEIFVGPRPEQYTWILKLLKTRKPGEIQPYKRKDLYPIK

SEQ ID NO: 13 - protein, 301 aa, Escherichia coli
MSMSHINYNHLYYFWHVYKEGSVVGAAEALYLTPQTITGQIRALEERLQGKLFKRKGRGLEPSELGELVYRYADKMFTLSQEMLDIVNYRKESNLLFDVGVADALSKRLVSSVLNAAVVEGEPIHLRCFESTHEMLLEQLSQHKLDMIISDCPIDSTQQEGLFSVRIGECGVSFWCTNPPPEKPFPACLEERRLLIPGRRSMLGRKLLNWFNSQGLNVEILGEFDDAALMKAFGAMHNAIFVAPTLYAYDFYADKTVVEIGRVENVMEEYHAIFAERMIQHPAVQRICNTDYSALFSPAVR

SEQ ID NO: 14 - protein, 949 aa, Escherichia coli
MKYLASFRTTLKASRYMFRALALVLWLLIAFSSVFYIVNALHQRESEIRQEFNLSSDQAQRFIQRTSDVMKELKYIAENRLSAENGVLSPRGRETQADVPAFEPLFADSDCSAMSNTWRGSLESLAWFMRYWRDNFSAAYDLNRVFLIGSDNLCMANFGLRDMPVERDTALKALHERINKYRNAPQDDSGSNLYWISEGPRPGVGYFYALTPVYLANRLQALLGVEQTIRMENFFLPGTLPMGVTILDENGHTLISLTGPESKIKGDPRWMQERSWFGYTEGFRELVLKKNLPPSSLSIVYSVPVDKVLERIRMLILNAILLNVLAGAALFTLARMYERRIFIPAESDALRLEEHEQFNRKIVASAPVGICILRTADGVNILSNELAHTYLNMLTHEDRQRLTQIICGQQVNFVDVLTSNNTNLQISFVHSRYRNENVAICVLVDVSSRVKMEESLQEMAQAAEQASQSKSMFLATVSHELRTPLYGIIGNLDLLQTKELPKGVDRLVTAMNNSSSLLLKIISDILDFSKIESEQLKIEPREFSPREVMNHITANYLPLVVRKQLGLYCFIEPDVPVALNGDPMRLQQVISNLLSNAIKFTDTGCIVLHVRADGDYLSIRVRDTGVGIPAKEVVRLFDPFFQVGTGVQRNFQGTGLGLAICEKLISMMDGDISVDSEPGMGSQFTVRIPLYGAQYPQKKGVEGLSGKRCWLAVRNASLCQFLETSLQRSGIVVTTYEGQEPTPEDVLITDEVVSKKWQGRAVVTFCRRHIGIPLEKAPGEWVHSVAAPHELPALLARIYLIEMESDDPANALPSTDKAVSDNDDMMILVVDDHPINRRLLADQLGSLGYQCKTANDGVDALNVLSKNHIDIVLSDVNMPNMDGYRLTQRIRQLGLTLPVIGVTANALAEEKQRCLESGMDSCLSKPVTLDVIKQTLTLYAERVRKSRDS

SEQ ID NO: 15 - protein, 61 aa, Escherichia coli
MLILTRRVGETLMIGDEVTVTVLGVKGNQVRIGVNAPKEVSVHREEIYQRIQAEKSQQSSY

SEQ ID NO: 16 - protein, 582 aa, Escherichia coli
MHNDKDLSTWQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 17 - protein, 337 aa, Escherichia coli
MTQENEIKRPIQDLEHEPIKPLDNSEKGSKVSQALETVTTTAEKVQRQPVIAHLIRATERFNDRLGNQFGAAITYFSFLSMIPILMVSFAAGGFVLASHPMLLQDIFDKILQNISDPTLAATLKNTINTAVQQRTTVGLVGLAVALYSGINWMGNLREAIRAQSRDVWERSPQDQEKFWVKYLRDFISLIGLLIALIVTLSITSVAGSAQQMIISALHLNSIEWLKPTWRLIGLAISIFANYLLFFWIFWRLPRHRPRKKALIRGTFLAAIGFEVIKIVMTYTLPSLMKSPSGAAFGSVLGLMAFFYFFARLTLFCAAWIATAEYKDDPRMPGKTQP

SEQ ID NO: 18 - protein, 306 aa, Escherichia coli
MFPQCKFSREFLHPRYWLTWFGLGVLWLWVQLPYPVLCFLGTRIGAMARPFLKRRESIARKNLELCFPQHSAEEREKMIAENFRSLGMALVETGMAWFWPDSRVRKWFDVEGLDNLKRAQMQNRGVMVVGVHFMSLELGGRVMGLCQPMMATYRPHNNQLMEWVQTRGRMRSNKAMIGRNNLRGIVGALKKGEAVWFAPDQDYGRKGSSFAPFFAVENVATTNGTYVLSRLSGAAMLTVTMVRKADYSGYRLFITPEMEGYPTDENQAAAYMNKIIEKEIMRAPEQYLWIHRRFKTRPVGESSLYI

SEQ ID NO: 19 - protein, 186 aa, Escherichia coli
MNVSKYVAIFSFVFIQLISVGKVFANADEWMTTFRENIAQTWQQPEHYDLYIPAITWHARFAYDKEKTDRYNERPWGGGFGLSRWDEKGNWHGLYAMAFKDSWNKWEPIAGYGWESTWRPLADENFHLGLGFTAGVTARDNWNYIPLPVLLPLASVGYGPVTFQMTYIPGTYNNGNVYFAWMRFQF

SEQ ID NO: 20 - protein, 547 aa, Escherichia coli
MLKRLLKRPSLNLLAWLLLAAFYISICLNIAFFKQVLQALPLDSLHNVLVFLSMPVVAFSVINIVLTLSSFLWLNRPLACLFILVGAAAQYFIMTYGIVIDRSMIANIIDTTPAESYALMTPQMLLTLGFSGVLAALIACWIKIKPATSRLRSVLFRGANILVSVLLILLVAALFYKDYASLFRNNKELVKSLSPSNSIVASWSWYSHQRLANLPLVRIGEDAHRNPLMQNEKRKNLTILIVGETSRAENFSLNGYPRETNPRLAKDNVVYFPNTASCGTATAVSVPCMFSDMPREHYKEELAQHQEGVLDIIQRAGINVLWNDNDGGCKGACDRVPHQNVTALNLPDQCINGECYDEVLFHGLEEYINNLQGDGVIVLHTIGSHGPTYYNRYPPQFRKFTPTCDTNEIQTCTKEQLVNTYDNTLVYVDYIVDKAINLLKEHQDKFTTSLVYLSDHGESLGENGIYLHGLPYAIAPDSQKQVPMLLWLSEDYQKRYQVDQNCLQKQAQTQHYSQDNLFSTLLGLTGVETKYYQAADDILQTCRRVSE

(19) United States
(12) Patent Application Publication
(10) Pub. No.: US 2022/0396761 A1
(43) Pub. Date: Dec. 15, 2022

(54) VIABLE BACTERIAL HOST CELL

(71) Applicant: INBIOSE N.V., Zwijnaarde (BE)

(72) Inventors: Joeri Beauprez, Zwijnaarde (BE); Sofie Maeseneire, Zwijnaarde (BE); Nico Snoeck, Zwijnaarde (BE)

(21) Appl. No.: 17/904,203

(22) PCT Filed: Feb. 12, 2021
(86) PCT No.: PCT/EP2021/053499

§ 371 Date: Aug. 12, 2022

(30) Foreign Application Priority Data
Feb. 14, 2020 (BE) BE2020/5095

(51) Int. Cl. C12N 1/20; C12N 15/90; C12N 15/52; C12N 15/70

(52) U.S. Cl. CPC C12N 1/205; C12N 15/90; C12N 15/52; C12N 15/70; C12Y 205/01055; C12Y 301/03045; C12R 2001/19

(57) ABSTRACT

The disclosure is in the technical field of synthetic biology and metabolic engineering. The disclosure provides engineered viable bacteria. In particular, the disclosure provides viable bacteria with mutated outer membrane biosynthetic pathway leading to disruption of the pathway, preferably substantially lacking lipopolysaccharide (LPS, endotoxin) within the outer membrane. The disclosure further provides methods of generating viable bacteria and uses thereof. The disclosure also provides compositions and methods for inducing immune responses and for researching and developing therapeutic agents. Furthermore, the disclosure is in the technical field of fermentation of metabolically engineered microorganisms producing bioproduct or metabolite.

### CROSS-REFERENCE TO RELATED APPLICATIONS

This application is a national phase entry under 35 U.S.C. § 371 of International Patent Application PCT/EP2021/053499, filed Feb. 11, 2021, designating the United States of America and published in English as International Patent Publication WO 2021/160829 A1 on Aug. 19, 2021, which claims the benefit under Article 8 of the Patent Cooperation Treaty to Belgian Patent Application Serial No. BE2020/5095, filed Feb. 14, 2020.

### STATEMENT ACCORDING TO 37 C.F.R. § 1.821(c) or (e)-SEQUENCE LISTING SUBMITTED AS A TXT AND PDF FILES

Pursuant to 37 C.F.R. § 1.821(c) or (e), files containing a TXT version and a PDF version of the Sequence Listing have been submitted concomitant with this application, the contents of which are hereby incorporated by reference.

### TECHNICAL FIELD

This disclosure is in the technical field of synthetic biology and metabolic engineering. The disclosure provides engineered viable bacteria. In particular, the disclosure provides viable bacteria with mutated outer membrane biosynthetic pathway leading to disruption of the pathway, preferably substantially lacking lipopolysaccharide (LPS, endotoxin) within the outer membrane. The disclosure further provides methods of generating viable bacteria and uses thereof. The disclosure also provides compositions and methods for inducing immune responses and for researching and developing therapeutic agents. Furthermore, the disclosure is in the technical field of fermentation of metabolically engineered microorganisms producing bioproduct or metabolite.

### BACKGROUND

Gram-negative bacteria (e.g., Escherichia co/i) have a cell wall, which consists of a double membrane enclosing a peptidoglycan containing periplasmatic space. The inner membrane (IM) or cytoplasmic membrane is a phospholipid bilayer whereas the outer membrane (OM) is asymmetrical. The OM of Gram-negative bacteria is also a lipid bilayer, but its surface exposed outer leaflet is composed of lipopolysaccharides or LPS. The outer membrane structure protects the bacteria against harsh environmental conditions and forms a barrier against numerous stress factors.

LPS, also called endotoxin, a major constituent of the outer membrane of Gram-negative bacteria, induces the mammal innate immune system. When infected, the uncontrolled growth of these bacteria can lead to the release of large amounts of LPS, which triggers a severe sceptic shock, damaging small blood vessels with fatal consequences for the host.

LPS is a glycophospholipid consisting of an antigenic, variable-size, carbohydrate chain covalently linked to lipid A, the conserved hydrophobic region structurally defined as N,O-acyl beta-1,6-D-glucosamine 1,4′-bisphosphate. Toxicity of LPS is expressed by lipid A through the interaction with B-cells and macrophages of the mammalian immune system, a process leading to the secretion of proinflammatory cytokines, mainly TNF, which may have fatal consequences for the host. Lipid A also activates human T-lymphocytes (Th-I) in vitro as well as murine CD4+ and CD8+ T-cell in vivo, a property that allows the host's immune system to mount a specific, anamnestic IgG antibody response to the variable-size carbohydrate chain of LPS. In order to fully express toxicity, LPS must retain its supramolecular architecture through the association of several units of glycophospholipid monomers forming the lipid A structure. This conformational rearrangement of the molecule is also fundamental for full expression of the immunogenic characteristic.

The hydrophobic anchor of LPS is a glucosamine-based phospholipid backbone called 2-keto 3-deoxy-D-manno-octulosonate (KDO)-LipidA. The biogenesis of KDO-LipidA involves nine enzymes and was first uncovered by Christian R. H. Raetz, hence this is called the Raetz pathway.

To create an expression host to be used in a biotechnological production process for the production of bioproducts it is desirable that it is devoid of the endotoxin LipidA as well as overacetylated Lipid IV_{A} constituents. U.S. Pat. No. 8,303,964 describes such cells, displaying only a tetraacylated Lipid IV_{A} outer membrane. To achieve this pure Lipid IV_{A} outer leaflet, all late acyltransferases (lpxL, lpxM, pagP, lpxP) and phosphoethanolamination (eptA), which are of major importance for the endotoxin immune response, were deleted in presence of the msbA148 suppressor mutation. This expression platform offers a way of making protein therapeutics while minimizing the cost for endotoxin purifications and elimination.

LPS structures are powerful adjuvants, but their use is severely limited by their endotoxic activity. By altering the biosynthesis pathway creating other acylated or dephosphatated variants, the reactogenicity can be strongly attenuated while retaining vaccine efficacy. For example, monophospho-lipidA (MPLA), lacking one of the two phosphate groups is less toxic than the original structure. MPLA is synthesized in Salmonella strains as vaccine adjuvant to elicit moderate immune response. In addition, also lipidA deficient strains could be used to eliminate the cost of LPS detoxification, which is otherwise necessary in e.g., the commercially available formulation of the meningococcal group B vaccine: Bexsero.

The creation of LPS variant and deficient strains also opens a new window for industrial bacteria, e.g., E. coli, for processes in feed, food and pharmaceutical productions as endotoxin levels need to be closely monitored, assessed and mitigated. For example, in a high cell density biotechnological production process, endotoxin levels can rise to millions of EU/mL in the supernatant, causing potential harm to humans and other mammals. In comparison, LPS levels in tap water only range from 1 to 20 EU/mL. To obtain a pure and safe product, these endotoxins must be removed. Removal of endotoxins is done using a wide range of techniques such as ultracentrifugation, two-phase extraction or affinity chromatography, but there is no universal removal technique, and all depends highly on the product type and application.

There is thus a need in the art to reduce the cost of downstream processing in respect of removal of outer membrane lipids, e.g., endotoxin.

### BRIEF SUMMARY

The disclosure provides a viable bacterial host cell. In particular, the disclosure provides viable bacteria mutated in the outer membrane synthesis pathway leading to disruption of the pathway and preferably substantially lacking lipopolysaccharide (LPS, endotoxin) within the outer membrane. The cell further comprises a mutation in the expression or the coding sequence of any one or more of the genes encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase. The disclosure further provides methods of generating viable, preferably non-toxic, bacteria and uses thereof. The disclosure also provides compositions and methods for inducing immune responses and for researching and developing therapeutic agents. The disclosure also provides methods for the production of a metabolite or a bioproduct employing the host cell of the disclosure.

The disclosure provides an alternative route to eliminate endotoxin formation. Elimination of these substances would then alleviate the need to measure, control and finally remove them in a biotechnological production process.

Embodiments of the disclosure provide a wide range of methods and compositions employing a bacterial host cell being mutated in the outer membrane synthesis pathway leading to disruption of the pathway. Further embodiments provide different uses of the bacterial host cell of the disclosure. Exemplary embodiments are described below in the Brief Summary, the Detailed Description and the Examples section below. The disclosure is not limited to these exemplary embodiments. The bacteria being mutated in the outer membrane synthesis pathway leading to disruption of the pathway may be generated by any mechanism. A diverse variety of different mechanisms for generating such bacteria are described herein. For example, in some embodiments, genes are mutated (e.g., so as to reduce or eliminate expression of functional protein) that are involved in KDO synthesis. In some embodiments, genes are mutated that are involved in association of KDO with Lipid IV_{A}. In some embodiments, genes are mutated that are involved in Lipid IV_{A} synthesis. In some embodiments, genes are mutated that are involved in LipidA synthesis. In some embodiments, other genes involved in LPS production or presentation are mutated.

The disclosure is not limited to gene mutation. In some embodiments, expression is altered using RNA interference or other techniques. In some embodiments, protein function is altered by providing inhibitors (e.g., synthetic or natural competitive or non-competitive ligands, antibodies, etc.). In some embodiments, modified bacteria are further supplied with nutrients, other modifications, or other components useful for maintaining health, growth, etc., in view of the alterations made to affect LPS status. Embodiments of the disclosure are not limited to these mechanisms unless specified otherwise. The disclosure demonstrates that bacteria lacking LPS are viable, may be made through a variety of routes, and find use in a variety of settings.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

The term “endogenous” within the context of the disclosure refers to any polynucleotide, polypeptide or protein sequence, which is a natural part of a cell and is occurring at its natural location in the cell chromosome. The term “exogenous” refers to any polynucleotide, polypeptide or protein sequence, which originates from outside the cell under study and is not a natural part of the cell or which is not occurring at its natural location in the cell chromosome or plasmid.

“Recombinant” means genetically engineered DNA prepared by transplanting or splicing genes from one species into the cells of a host organism of a different species. Such DNA becomes part of the host's genetic make-up and is replicated. “Mutant” cell or microorganism as used within the context of the disclosure refers to a cell or microorganism, which is genetically engineered or has an altered genetic make-up.

The term “modified expression” of a gene relates to a change in expression compared to the wild type expression of the gene in any phase of the production process of the sialylated oligosaccharide. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression or reduced expression is obtained by means of common well-known technologies for a skilled person (such as the usage of siRNA, CRISPR, CRISPRi, riboswitches, recombineering, homologous recombination, ssDNA mutagenesis, RNAi, miRNA, asRNA, mutating genes, knocking-out genes, transposon mutagenesis, etc.), which are used to change the genes in such a way that they are less-able (i.e., statistically significantly ‘less-able’ compared to a functional wild-type gene) or completely unable (such as knocked-out genes) to produce functional final products. Lower expression or reduced expression can, for instance, be obtained by mutating one or more base pairs in the promoter sequence or changing the promoter sequence fully to a constitutive promoter with a lower expression strength compared to the wild type or an inducible promoter, which result in regulated expression or a repressible promoter, which results in regulated expression. Overexpression or expression is obtained by means of common well-known technologies for a skilled person, wherein e.g., the gene is part of an “expression cassette,” which relates to any sequence in which a promoter sequence, untranslated region sequence (UTR) (containing either a ribosome binding sequence or Kozak sequence), a coding sequence (for instance, a membrane protein gene sequence) and optionally a transcription terminator is present, and leading to the expression of a functional active protein. The expression is either constitutive or conditional or regulated.

The term “wild type” refers to the commonly known genetic or phenotypical situation as it occurs in nature.

A “riboswitch” as used herein is defined to be part of the messenger RNA that folds into intricate structures that block expression by interfering with translation. Binding of an effector molecule induces conformational change(s) permitting regulated expression post-transcriptionally.

As used herein, the terms “subject” and “patient” refer to any animal, such as a mammal like a dog, cat, bird, livestock, and preferably a human.

As used herein, the terms “LPS related disorder,” “condition associated with endotoxin,” “endotoxin associated disorder,” “endotoxin-related disorder,” “sepsis,” “sepsis related disorder,” or similar terms, describe any condition associated with LPS, e.g., a condition associated with bacteraemia or introduction of lipopolysaccharide into the blood stream or onto an extra-gastrointestinal mucosal surface (e.g., the lung). Such disorders include, but are not limited to, endotoxin-related shock, endotoxin-related disseminated intravascular coagulation, endotoxin-related anaemia, endotoxin-related thrombocytopenia, endotoxin-related adult respiratory distress syndrome, endotoxin-related renal failure, endotoxin-related liver disease or hepatitis, systemic immune response syndrome (SIRS) resulting from Gram-negative infection, Gram-negative neonatal sepsis, Gram-negative meningitis, Gram-negative pneumonia, neutropenia and/or leukopenia resulting from Gram-negative infection, hemodynamic shock and endotoxin-related pyrosis.

The term “viable non-toxic Gram-negative bacteria” refers to a viable Gram-negative bacterial strain comprising an outer membrane substantially free of LPS.

The terms “cells” and “host cells” and “recombinant host cells,” which are used interchangeably herein, refer to cells that are capable of or have been transformed with a vector, typically an expression vector. The host cells used herein are cells comprising an outer membrane. Such cells can be Gram-negative bacterial cells. It is understood that such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The term “defective” as used herein, with regard to a gene or gene expression, means that the gene is not a wildtype gene and that the organism does not have a wildtype genotype and/or a wildtype phenotype. The defective gene, genotype or phenotype may be the consequence of a mutation in that gene, or of a gene that regulates the expression of that gene (e.g., transcriptional or post-transcriptional), such that its normal expression is disrupted or extinguished. “Disrupted gene expression” is intended to include both complete inhibition and decreased gene expression (e.g., as in a leaky mutation), below wildtype gene expression.

The terms “conditional,” “regulated,” “inducible,” or similar terms, refer, in particular, to gene expression, which is not constitutive, but which takes place in response to a stimulus (e.g., temperature, heavy metals or other medium additive).

The term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “pyrogenic” or “pyrogenicity” refers to the ability of a compound to induce fever or a febrile response when administered to a subject. Such febrile responses are generally mediated by the host proinflammatory cytokines IL-I, IL-6 and/or TNF-a, the secretion of which is induced, e.g., by LPS.

As used herein, the term “transfection” means the introduction of a nucleic acid (e.g., via an expression vector) into a recipient cell by nucleic acid-mediated gene transfer.

“Transformation,” as used herein, refers to a process in which a cell's genotype is changed as a result of the cellular uptake of exogenous DNA or RNA. A transformed cell can also be one that expresses a nucleic acid that interferes with the expression of an endogenous nucleic acid.

As used herein, the term “transgene” means a nucleic acid that has been introduced into a cell. A transgene could be partly or entirely heterologous, i.e., foreign, to the transgenic cell into which it is introduced, or, can be homologous to an endogenous gene of the cell into which it is introduced, but which is designed to be inserted, or is inserted, into the cell's genome in such a way as to alter the genome of the cell into which it is inserted. A transgene can also be present in a cell in the form of an episome.

The term “treating” a subject for a condition or disease, as used herein, is intended to encompass curing, as well as ameliorating at least one symptom of the condition or disease.

The term “vector” refers to a nucleic acid molecule, which is capable of transporting another nucleic acid to which it has been linked. Vectors capable of directing the expression of genes to which they are operatively linked are referred to herein as “expression vectors.”

The term “expression system” as used herein refers to an expression vector under conditions whereby an mRNA may be transcribed and/or an mRNA may be translated into protein, structural RNA, or other cellular component. The expression system may be an in vitro expression system, which is commercially available or readily made according to art known techniques, or may be an in vivo expression system, such as a eukaryotic or prokaryotic cell containing the expression vector. In general, expression vectors of utility in recombinant DNA techniques are often in the form of “plasmids,” which refer generally to circular double stranded DNA loops that, in their vector form, are not bound to the chromosome. In the present specification, “plasmid” and “vector” are used interchangeably as the plasmid is the most commonly used form of vector. However, the disclosure is intended to include such other forms of expression vectors, which serve equivalent functions and are well known in the art or which become known in the art subsequently hereto (e.g., cosmid, phagemid and bacteriophage vectors).

The term “outer membrane” as used herein refers to the outer boundary or membrane of a cell comprising a double membrane. Gram-negative bacteria (e.g., Escherichia coli) have a cell wall, which consists of a double membrane enclosing a peptidoglycan containing periplasmatic space. The inner membrane (IM) or cytoplasmic membrane is a phospholipid bilayer whereas the outer membrane (OM) is asymmetrical. The OM of Gram-negative bacteria is also a lipid bilayer, but its surface exposed outer leaflet is composed of lipopolysaccharides or LPS. The outer membrane structure protects the bacteria against harsh environmental conditions and forms a barrier against numerous stresses.

The terms bioproduct and metabolite as used herein is any product that can be synthesized in a biological manner, i.e., via enzymatic conversion, microbial biosynthesis, cellular biosynthesis.

Examples of bioproducts and metabolites are:

1) Small organic molecules, such as but not limited to organic acids, alcohols, amino acids; proteins, such as but not limited to enzymes, antibodies, single cell protein, nutritional proteins, albumins, lactoferrin, glycolipids and glycopeptides; antibiotics, such as but not limited to antimicrobial peptides, polyketides, penicillins, cephalosporins, polymyxins, rifampycins, lipiarmycins, quinolones, sulfonamides, macrolides, lincosamides, tetracyclines, aminoglycosides cyclic lipopeptides (such as daptomycin), glycylcyclines (such as tigecycline), oxazolidinones (such as linezolid), lipiarmycins fidaxomicin; lipids, such as but not limited to arachidonic acid, docosahexaenic acid, linoleic acid, Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Tetracosapentaenoic acid, Tetracosahexaenoic acid (Nisinic acid); Flavonoids, glycolipids, ceramides, sphingolipids, carbohydrates, monosaccharides, disaccharides, polysaccharides, oligosaccharides such as but not limited to human milk oligosaccharides, glycosaminoglycans, chitosans, chondrotoines, heparosans, Glucuronylated oligosaccharides, fucosylated oligosaccharides, neutral oligosaccharide and/or sialylated oligosaccharides;

2) A human milk oligosaccharide, such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide and/or sialylated milk oligosaccharides;

3) A ‘sialylated oligosaccharide,’ a charged sialic acid containing oligosaccharide, i.e., an oligosaccharide having a sialic acid residue. It has an acidic nature. Some examples are 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Aca-2,3Gal beta-1,3GalNac beta-1,3Gala-1,4Gal beta-1,4Gal), sialylated tetrasaccharide (Neu5Aca-2,3Gal beta-1,4GlcNac beta-14GlcNAc), pentasaccharide LSTD (Neu5Aca-2,3Gal beta-1,4GlcNac beta-1,3Gal beta-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′sialyllactose, Neu5Aca-2,3Gal β-4Glc) and oligosaccharides comprising the GM3 motif, GD3 Neu5Aca-2,8Neu5Aca-2,3Gal β-1,4Glc GT3 (Neu5Aca-2,8Neu5Aca-2,8Neu5Aca-2,3Gal β-1,4Glc); GM2 GalNAc β-1,4(Neu5Aca-2,3)Gal β-1,4Glc, GM1 Gal β-1,3GalNAc β-1,4(Neu5Aca-2,3)Gal β-1,4Glc, GD1a Neu5Aca-2,3Gal β-1,3GalNAc β-1,4(Neu5Aca-2,3)Gal β-1,4Glc GT1a Neu5Aca-2,8Neu5Aca-2,3Gal β-1,3GalNAc β-1,4(Neu5Aca-2,3)Gal β-1,4Glc GD2 GalNAc β-1,4(Neu5Aca-2,8Neu5Aca2,3)Gal β-1,4Glc GT2 GspalNAc β-1,4(Neu5Aca-2,8Neu5Aca-2,8Neu5Aca2,3)Gal β-1,4Glc GD1b, Gal β-1,3GalNAc β-1,4(Neu5Aca-2,8Neu5Aca2,3)Gal β-1,4Glc GT1b Neu5Aca-2,3Gal β-1,3GalNAc β-1,4(Neu5Aca-2,8Neu5Aca2,3)Gal β-1,4Glc GQ1b Neu5Aca-2,8Neu5Aca-2,3Gal β-1,3GalNAc β-1,4(Neu5Aca-2,8Neu5Aca2,3)Gal β-1,4Glc GT1c Gal β-1,3GalNAc β-1,4(Neu5Aca-2,8Neu5Aca-2,8Neu5Aca2,3)Gal β-1,4Glc GQ1c, Neu5Aca-2,3Gal β-1,3GalNAc β-1,4(Neu5Aca-2,8Neu5Aca-2,8Neu5Aca2,3)Gal β-1,4Glc GP1c Neu5Aca-2,8Neu5Aca-2,3Gal β-1,3GalNAc β-1,4(Neu5Aca-2,8Neu5Aca-2,8Neu5Aca2,3)Gal β-1,4Glc GD1a Neu5Aca-2,3Gal β-1,3(Neu5Aca-2,6)GalNAc β-1,4Gal β-1,4Glc Fucosyl-GM1 Fuca-1,2Gal β-1,3GalNAc β-1,4(Neu5Aca-2,3)Gal β-1,4Glc; all of which may be extended to the production of the corresponding gangliosides by reacting the above oligosaccharide moieties with ceramide or synthetizing the above oligosaccharides on a ceramide;

4) A ‘fucosylated oligosaccharide,’ generally understood in the state of the art as an oligosaccharide that is carrying a fucose-residue. Examples comprise 2′-fucosyllactose, 3-fucosyllactose, difucosyllactose, lactodifucotetraose (LDFT), Lacto-N-fucopentaose I (LNF I), Lacto-N-fucopentaose II (LNF II),), Lacto-N-fucopentaose III (LNF III), lacto-N-fucopentaose V (LNF V), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose;

5) A ‘neutral oligosaccharide,’ generally understood in the state of the art as an oligosaccharide that has no negative charge originating from a carboxylic acid group. Examples of such neutral oligosaccharide are 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose;

6) A monosaccharide preferably selected from the group comprising Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

7) A disaccharide or oligosaccharide containing any one or more monosaccharide as described herein.

The term polyol as used herein is an alcohol containing multiple hydroxyl groups. For example, glycerol, sorbitol, or mannitol.

The term “sialic acid” as used herein refers to the group comprising sialic acid, neuraminic acid, N-acetylneuraminic acid and N-Glycolylneuraminic acid.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using BLAST and PSI-BLAST (Altschul et al., 1990, J Mol Biol 215:3, 403-410; Altschul et al., 1997, Nucleic Acids Res 25: 17, 3389-402). For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

An amino acid sequence or polypeptide sequence or protein sequence, used herein interchangeably, of the polypeptide used herein can be a sequence as indicated with the SEQ ID NO of the attached sequence listing. The amino acid sequence of the polypeptide can also be an amino acid sequence that has 80% or more sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of the respective SEQ ID NO.

### BRIEF DESCRIPTION OF THE DRAWINGS

FIG. 1 shows a schematic representation of the biosynthesis and incorporation of KDO into LPS.

### DETAILED DESCRIPTION

The disclosure provides a bacterial host cell. In particular, the disclosure provides a viable bacterial host cell that can grow and divide with a mutated outer membrane biosynthetic pathway leading to disruption of the pathway. The cell comprises a mutation in the expression or the coding sequence of any one or more of the genes encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase. Preferably such cell is a Gram-negative bacterial cell (e.g., E. coli) substantially lacking lipopolysaccharide (LPS, endotoxin) within the outer membrane. The disclosure further provides methods of generating a viable bacterial cell, preferably a non-toxic bacterial cell, e.g., a non-toxic Gram-negative bacterial cell, and uses thereof. The disclosure also provides compositions and methods for inducing immune responses and for researching and developing therapeutic agents. The disclosure also provides methods for the production of a bioproduct or metabolite using the bacterial host of the disclosure.

The disrupted outer membrane biosynthesis pathway provides a low outer membrane permeability, which can advantageously be used for transforming providing high transformation efficiency cells. E.g., the bacterial host cell of the disclosure can be transformed with DNA plasmids during the generation of DNA libraries. The bacterial host cell being a high transformation efficiency cell is thus a useful host for all recombination DNA technologies.

In a first embodiment of the disclosure, the disrupted outer membrane biosynthetic pathway of the host cell is the KDO₂-LipidA biosynthetic pathway.

In an exemplary embodiment thereof, the host cell lacks KDO in the outer membrane.

In a further embodiment, the host cell is substantially free of LPS expression.

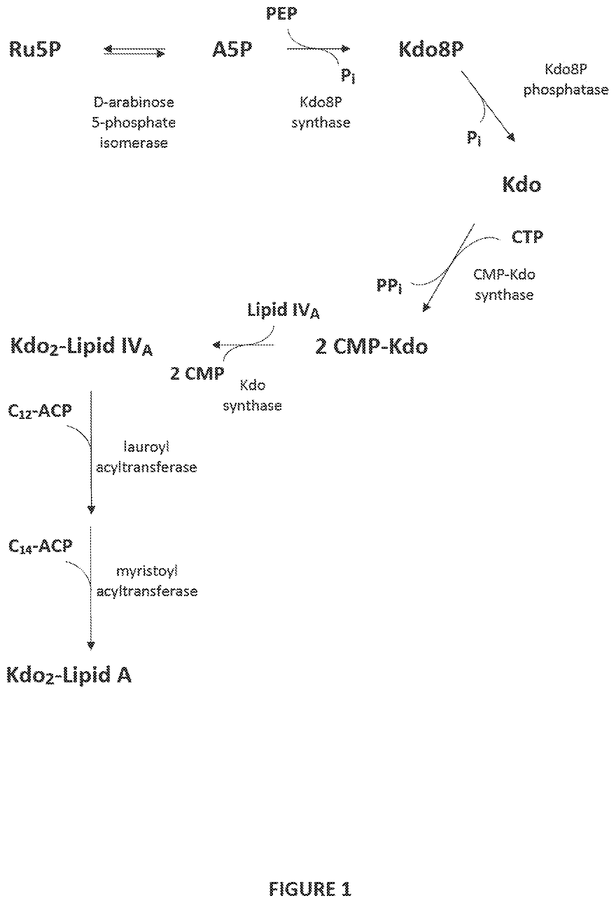

LPS is an immunostimulatory and/or inflammatory molecule recognized as a mediator of Gram-negative pathogenesis and generalized inflammation. As such the term endotoxin is often used interchangeably with LPS. The LPS layer is essential to both the form and function of the OM of Gram-negative bacteria, providing Gram-negative pathogenesis and survival of the bacterium. LPS of various Gram-negative bacteria is built conform to a common structural architecture conceptually comprising among others: the outer membrane embedded lipid A. Lipid A is the most conserved LPS domain amongst Gram-negative bacterial genera, and, being the structural component responsible for the biological activities within the host, represents an endotoxic principle of LPS. The majority of Gram-negative bacteria elaborate an inner core containing at least one 2-keto 3-deoxy-D-manno-octulosonate (KDO) molecule. KDO is an essential component of LPS that is a conserved residue found in nearly all LPS structures. The minimal LPS structure required for growth of E. coli is made of two KDO residues attached to lipid A (KDO₂-lipidA), indicating the importance of KDO in maintaining the integrity and viability of the bacterial cell. As schematically depicted in FIG. 1, the biosynthesis pathway of LPS structures is initiated by the enzyme d-arabinose 5-phosphate (A5P) isomerase (API), which converts the pentose pathway intermediate D-ribulose 5-phosphate into A5P. Subsequently, A5P is condensed with phosphoenolpyruvate to form KDO 8-phosphate (KDO8P) by KDO 8-phosphate synthase, hydrolysed to KDO by KDO 8-phosphate phosphatase, activated as the sugar nucleotide CMP-KDO by a CMP-KDO synthase, before finally being transferred from CMP-KDO to the acceptor lipid IV_{A} by a KDO synthase. The lauroyl and myristoyl-acyl carrier protein dependent acyltransferases transfer the fatty acids laurate and myristate, respectively, to KDO₂-lipid IV_{A} to form the characteristic acyloxyacyl units of hexaacylated KDO-lipid A.

In a further embodiment, the host cell can further display LipidA in the outer membrane.

Preferably the disruption in the outer membrane biosynthetic pathway is caused by at least one mutation in a gene encoding a protein selected from the group consisting of D-arabinose 5-phosphate isomerase, KDO8P synthase, CMP-KDO synthetase, KDO8P phosphatase and KDO transferase. Such mutation can be a mutation in the expression or the coding sequence of the genes encoding the listed proteins. Such mutation preferably provides a reduced expression or an abolished expression of one or more genes encoding such D-arabinose 5-phosphate isomerase, KDO8P synthase, CMP-KDO synthetase, KDO8P phosphatase and/or KDO transferase.

Preferably, the disruption in the outer membrane biosynthetic pathway is caused by at least one mutation in a gene or expression of a gene encoding for the protein selected from the group consisting of gutQ, kdsD, kdsA, kdsB, kdsC and waaA, wherein gutQ and kdsD encode a D-arabinose 5-phosphate isomerase, kdsA encodes a KDO8P synthase, kdsB encodes a CMP-KDO synthetase, kdsC encodes a KDO8P phosphatase and waaA encodes a KDO transferase.

In another embodiment, the viable host cell with mutated outer membrane biosynthetic pathway leading to disruption of the pathway, comprises a mutation in the expression or the coding sequence of any one or more of the genes encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase and displays LipidIV_{A} in the outer membrane. Such cell is additionally mutated in at least one gene or the expression of at least one gene encoding for a protein selected from the group consisting of lauroyl acyltransferase and myristoyl-acyl carrier protein-dependent acyltransferase. Such mutation can be a mutation in the expression or the coding sequence of the genes encoding the listed proteins. Such mutation preferably provides a reduced expression or an abolished expression of one or more genes encoding such lauroyl acyltransferase and myristoyl-acyl carrier protein-dependent acyltransferase. Preferably, the host cell is additionally mutated in at least one gene or the expression of at least one gene selected from the group consisting of LpxL and/or LpxM.

In a further embodiment of the disclosure, the poly-β-1,6-N-acetyl-D-glucosamine outer membrane porin is encoded by pgaA, poly-β-1,6-N-acetyl-D-glucosamine N-deacetylase is encoded by pgaB, and/or poly-N-acetyl-D-glucosamine synthase activity is encoded by pgaD and pgaC.

In a further embodiment of the disclosure, the mutation in the expression or the coding sequence of any one or more of the genes encoding for the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase provides poly-N-acetylglucosamine overproduction. Preferably, the poly-N-acetylglucosamine overproduction is provided by a mutation in the pgaABCD cluster. In another preferred embodiment, the poly-N-acetylglucosamine overproduction is provided by overexpression or introducing and expressing, preferably overexpressing, any one or more genes encoding for the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase.

In a further embodiment, at least two of the genes encoding for the proteins selected from an poly-β-1,6-N-acetyl-D-glucosamine outer membrane porin, poly-β-1,6-N-acetyl-D-glucosamine N-deacetylase, and/or poly-N-acetyl-D-glucosamine synthase, are clustered in an operon.

In another preferred embodiment, the poly-N-acetylglucosamine overproduction is provided by overexpression or introducing and expressing, preferably overexpressing, any one or more genes encoding for the proteins selected from a Na⁺/H⁺ antiporter regulator and a sensor histidine kinase.

As used herein, preferably the Na⁺/H⁺ antiporter regulator is an nhaR protein, preferably from E. coli. In another preferred embodiment, the Na⁺/H⁺ antiporter regulator comprises a sequence as given by SEQ ID NO:13 or a sequence having 80% or more sequence identity to the sequence as given by SEQ ID NO:13 and having Na⁺/H⁺ antiporter regulator activity.

As used herein, preferably the sensor histidine kinase is an resC protein, preferably from E. coli. In another preferred embodiment, the sensor histidine kinase comprises a sequence as given by SEQ ID NO:14 or a sequence having 80% or more sequence identity to the sequence as given by SEQ ID NO:14 and having sensor histidine kinase activity.

In another preferred embodiment, the poly-N-acetylglucosamine overproduction is provided by a reduced or abolished expression of a carbon storage regulator encoding gene or by a reduced or abolished expression of a carbon storage regulator.

As used herein, preferably the carbon storage regulator is a csrA, preferably from E. coli. In another preferred embodiment, the carbon storage regulator comprises a sequence as given by SEQ ID NO:15, or a sequence having 80% or more sequence identity to the sequence as given by SEQ ID NO:15 and having carbon storage regulator activity.

It was surprisingly found that an overproduction of poly-N-acetylglucosamine overcomes reduced or completely abolished expression of at least one essential gene for outer membrane production.

In an embodiment of the disclosure, the mutation in the expression or the coding sequences of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase provides an increased activity of at least one of the proteins, preferably an overexpression of at least one, two, three or four of the proteins, more preferably an overexpression of the operon encoding the proteins. In another preferred embodiment, the increased activity of at least one of the proteins is provided by introducing and expressing any one or more of the genes encoding for the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase. Preferably, the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is an overexpression of the pgaABCD cluster. Alternatively, a pgaABCD cluster can be introduced and expressed in the host cell with disrupted outer membrane biosynthetic pathway. It was surprisingly found that an overexpression of the pgaABCD overcomes reduced or completely abolished expression of at least one essential gene for outer membrane production.

In another preferred embodiment, the increased activity of at least one of the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase is provided by overexpression or introducing and expressing, preferably overexpressing, any one or more genes encoding for the proteins selected from a Na⁺/H⁺ antiporter regulator and a sensor histidine kinase. Preferably the Na⁺/H⁺ antiporter regulator is an nhaR protein, preferably from E. coli. Preferably the sensor histidine kinase is an resC protein, preferably from E. coli.

In another preferred embodiment, the increased activity of at least one of the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase is provided by a reduced or abolished expression of a carbon storage regulator. Preferably the carbon storage regulator is a csrA, preferably from E. coli.

In a preferred embodiment of the disclosure, the host cell further comprises a mutation, which eliminates association between KDO and LipidIV_{A}.

Preferably, an isolated viable bacterial host cell is provided, wherein the cell comprises a first mutation leading to a disruption of the KDO₂-LipidA biosynthetic pathway, and a second mutation in a gene selected from the pgaABCD cluster, and wherein the bacterial cell lacks KDO and displays LipidA in its outer membrane. Such first mutation can reside in a gene selected from the group consisting of gutQ, kdsD, kdsA, kdsB, and waaA. In an alternative preferred embodiment, an isolated viable bacterial host cell is provided wherein the cell comprises a first mutation leading to a disruption of the KDO₂-LipidA biosynthetic pathway, and a second mutation in a gene selected from the pgaABCD cluster, and wherein the bacterial cell lacks KDO and displays LipidIV_{A} in its outer membrane. The first mutation in this embodiment can reside in a gene selected from the group consisting of gutQ, kdsD, kdsA, kdsB, and waaA and the cell further comprises at least one mutation in a gene selected from the group consisting of LpxL and LpxM.

According to a preferred embodiment, the disruption in the outer membrane biosynthetic pathway is caused by at least one mutation in the gene encoding a KDO transferase. Preferably, the disruption in the outer membrane biosynthetic pathway is caused by at least one mutation resulting in a lack of expression of waaA.

Alternatively or preferably, the disruption in the outer membrane biosynthetic pathway results in a lack of expression of D-arabinose 5-phosphate isomerase (API).

The disclosure is not limited to a particular method of suppressing API protein expression. The disclosure is not limited to a particular method of suppressing API expression. In some embodiments, API expression is suppressed through suppression of KDO protein expression. The disclosure is not limited to a particular method of suppressing KDO protein expression. In some embodiments, KDO protein expression is suppressed through, for example, mutation of a D-arabinose 5-phosphate isomerase, a KDO8P synthase, a CMP-KDO synthetase, a KDO8P phosphatase and/or a KDO transferase, an ATP-dependent translocator, an inner membrane protein, (e.g. gutQ, kdsD, kdsA, kdsB, waaA, msbA, and/or yhjD gene) or mutations of any other biosynthetic, processing, or trafficking genes.

In some embodiments, mutations of the D-arabinose 5-phosphate isomerase genes inhibit API expression within the bacterial cell, which inhibits KDO expression, which inhibits outer membrane LPS expression. In some embodiments, the disclosure provides a viable bacterial host cell further mutated in the KDO8P synthase (e.g., kdsA) gene, CMP-KDO synthetase (e.g., kdsB) gene or KDO transferase (e.g., waaA) gene. Experiments conducted in the art already showed that mutations of kdsA, kdsB, and/or waaA inhibit the LPS biosynthetic pathway by preventing production of KDO or association between KDO₂ and Lipid IV_{A} such that Lipid IV_{A} alone is transported to the outer membrane. The bacterial cells survive and are LPS free and non-toxic.

According to the disclosure, the cell can further comprise a mutation in the expression or the coding sequence of a gene selected from an ATP-dependent translocator, inner membrane protein, D-arabinose 5-phosphate isomerase, Palmitoleoyl acyltransferase, Lipid A palmitoyltransferase, and/or phosphoethanolamine transferase, preferably the ATP-dependent translocator is an ATP-dependent Lipid A-core flippase; preferably the inner membrane protein is a transmembrane transporter. Such mutation providing preferably a reduced or an abolished expression of one or more genes encoding the ATP dependent translocator, inner membrane protein, D-arabinose 5-phosphate isomerase, Palmitoleoyl acyltransferase, Lipid A palmitoyltransferase, and/or phosphoethanolamine transferase. Preferably, the cell comprises a mutation in the expression or the coding sequence of a gene selected from msbA, yhjD, gutQ, kdsD, lpxP, pagP, eptA. The modifications may be effected by point mutations, deletions, insertions, expression of antisense RNA, etc., providing modified or deleted genes that encode one or more protein required for biosynthesis of a lipopolysaccharide (e.g., preferably the gutQ gene, the kdsD gene, the lpxA gene, the pagP gene, the lpxP gene, and the eptA gene).

According to the disclosure, the poly-β-1,6-N-acetyl-D-glucosamine outer membrane porin is preferably a polypeptide sequence comprising a sequence as given by SEQ ID NO:7 or a sequence having 80% or more sequence identity to the sequence as given by SEQ ID NO:7 and having poly-β-1,6-N-acetyl-D-glucosamine transmembrane transporter activity, poly-β-1,6-N-acetyl-D-glucosamine N-deacetylase is preferably a polypeptide sequence comprising a sequence as given by SEQ ID NO:8 or a sequence having 80% or more sequence identity to the sequence as given by SEQ ID NO:8 and having poly-β-1,6-N-acetyl-D-glucosamine N-deacetylase activity, and/or poly-N-acetyl-D-glucosamine synthase is preferably a polypeptide sequence comprising a sequence as given by any one of SEQ ID NO:9 or SEQ ID NO:10 or a sequence having 80% or more sequence identity to the sequence as given by any one of SEQ ID NO:9 or SEQ ID NO:10 and having poly-N-acetyl-D-glucosamine synthase activity.

Further according to the disclosure, the ATP-dependent translocator is preferably a polypeptide sequence comprising a sequence as given by SEQ ID NO:16 or a sequence having 80% or more sequence identity to the sequence as given by SEQ ID NO:16 and having ATP-dependent translocator activity; the inner membrane protein is preferably a polypeptide sequence comprising a sequence as given by SEQ ID NO:17 or a sequence having 80% or more sequence identity to the sequence as given by SEQ ID NO:17 and having transmembrane transporter activity, lauroyl acyltransferase is preferably a polypeptide sequence comprising a sequence as given by SEQ ID NO:11 or a sequence having 80% or more sequence identity to the sequence as given by SEQ ID NO:11 and having lauroyl acyltransferase activity; D-arabinose 5-phosphate isomerase is preferably a polypeptide sequence comprising a sequence as given by any one of SEQ ID NO:2 or SEQ ID NO:1 or a sequence having 80% or more sequence identity to the sequence as given by any one of SEQ ID NO:2 or SEQ ID NO:1 and having D-arabinose 5-phosphate isomerase activity, Palmitoleoyl acyltransferase is preferably a polypeptide sequence comprising a sequence as given by SEQ ID NO:18 or a sequence having 80% or more sequence identity to the sequence as given by SEQ ID NO:18 and having Palmitoleoyl acyltransferase activity, Lipid A palmitoyltransferase is preferably a polypeptide sequence comprising a sequence as given by SEQ ID NO:19 or a sequence having 80% or more sequence identity to the sequence as given by SEQ ID NO:19 and having Lipid A palmitoyltransferase activity, phosphoethanolamine transferase is preferably a polypeptide sequence comprising a sequence as given by SEQ ID NO:20 or a sequence having 80% or more sequence identity to the sequence as given by SEQ ID NO:20 and having phosphoethanolamine transferase activity, myristoyl-acyl carrier protein-dependent acyltransferase is preferably a polypeptide sequence comprising a sequence as given by SEQ ID NO:12 or a sequence having 80% or more sequence identity to the sequence as given by SEQ ID NO:12 and having myristoyl-acyl carrier protein-dependent acyltransferase activity, KDO8P synthase is preferably a polypeptide sequence comprising a sequence as given by SEQ ID NO:3 or a sequence having 80% or more sequence identity to the sequence as given by SEQ ID NO:3 and having KDO8P synthase activity, CMP-KDO synthetase is preferably a polypeptide sequence comprising a sequence as given by SEQ ID NO:4 or a sequence having 80% or more sequence identity to the sequence as given by SEQ ID NO:4 and having CMP-KDO synthetase activity, KDO transferase is preferably a polypeptide sequence comprising a sequence as given by SEQ ID NO:6 or a sequence having 80% or more sequence identity to the sequence as given by SEQ ID NO:6 and having KDO transferase activity, KDO8P phosphatase is preferably a polypeptide sequence comprising a sequence as given by SEQ ID NO:5 or a sequence having 80% or more sequence identity to the sequence as given by SEQ ID NO:5 and having KDO8P phosphatase activity.

The host cell as used herein can be a Gram-negative bacterium. Preferably, the cell is a Gram-negative bacterial cell. The disclosure furthermore contemplates the use of any type of Gram-negative bacterial cell in the construction of viable Gram-negative bacteria substantially lacking outer membrane LPS expression. Examples of Gram-negative bacteria useful in the disclosure include, but are not limited to, Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Citrobacter spp., Chlamydia spp., Brucella spp., Pseudomonas spp., Helicobacter spp., Moraxella spp., Stenotrophomonas spp., Bdellovibrio spp., Acinetobacter spp., Enterobacter spp. and Vibrio spp. Even more preferably, the host cell is selected from Escherichia spp., Salmonella spp., and Pseudomonas spp. In preferred embodiments, Escherichia coli is used. Examples of Escherichia strains, which can be used include, but are not limited to, Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the disclosure specifically relates to a mutated and/or transformed Escherichia coli strain as indicated above wherein the E. coli strain is a K12 strain. More specifically, the disclosure relates to a mutated and/or transformed Escherichia coli strain as indicated above wherein the K12 strain is E. coli MG1655.

Alternatively, the E. coli is selected from the group consisting of K-12 strain, W3110, MG1655, B/r, BL21, O157:h7, 042, 101-1, 1180, 1357, 1412, 1520, 1827-70, 2362-75, 3431, 53638, 83972, 929-78, 98NK2, ABU 83972, B, B088, B171, B185, B354, B646, B7A, C, c7122, CFT073, DH1, DH5a, E110019, E128010, E74/68, E851/71, EAEC 042, EPECa11, EPECa12, EPECa14, ETEC, H10407, F11, F18+, FVEC1302, FVEC1412, GEMS_EPEC1, HB101, HT115, KO11, LF82, LT-41, LT-62, LT-68, MS107-1, MS119-7, MS124-1, MS 145-7, MS 79-2, MS 85-1, NCTC 86, Nissle 1917, NT:H19, NT:H40, NU14, O103:H2, O103:HNM, O103:K+, O104:H12, O108:H25, O109:H9, O111H−, O111:H19, O111:H2, O111:H21, O111:NM, O115:H−, O115:HMN, O115:K+, O119:H6, O119:UT, O124:H40, O127a:H6, O127:H6, O128:H2, O131:H25, O136:H−, O139:H28 (strain E24377A/ETEC), O13:H11, O142:H6, O145:H−, O153:H21, O153:H7, O154:H9, O157:12, O157:H−, O157:H12, O157:H43, O157:H45, O157:H7 EDL933, O157:NM, O15:NM, O177:H11, O17:K52:H18 (strain UMN26/ExPEC), O180:H−, O1:K1/APEC, O26, O26:H−, O26:H11, O26:H1L:K60, O26:NM, O41:H−, O45:K1 (strain S88/ExPEC), O51:H−, O55:H51, O55:H6, O55:H7, O5:H−, O6, O63:H6, O63:HNM, O6:K15:H31 (strain 536/UPEC), O7:K1 (strain IAI39/ExPEC), O8 (strain IAI1), O81 (strain ED1a), O84:H−, O86a:H34, O86a:H40, O90:H8, O91:H21, O9:H4 (strain HS), O9:H51, ONT:H−, ONT:H25, OP50, Orough:H12, Orough:H19, Orough:H34, Orough:H37, Orough:H9, OUT:H12, OUT:H45, OUT:H6, OUT:H7, OUT:HNM, OUT:NM, RN587/1, RS218, 55989/EAEC, B/BL21, B/BL21-DE3, SE11, SMS-3-5/SECEC, UTI89/UPEC, TA004, TA155, TX1999, and Vir68.

In some embodiments, the disclosure provides a viable Gram-negative bacterial host cell lacking KDO despite exclusively elaborating the endotoxically inactive LPS precursor lipid IV_{A}, a known antagonist of LPS-induced sepsis in humans. In alternative embodiments, the disclosure provides viable Gram-negative bacterial host cells lacking KDO and displaying lipidA in the outer membrane. In some embodiments, the disclosure provides viable Gram-negative bacteria lacking D-arabinose 5-phosphate isomerase (API) expression. In some embodiments, the viable Gram-negative bacterial host cell comprises mutations such that the cell is substantially free of KDO. In some embodiments, the mutations include one or more mutations in one or more genes involved in KDO synthesis or modification. In some embodiments, the viable Gram-negative bacterial host cell comprises mutations wherein the mutations prevent association between KDO and Lipid IV_{A} in the LPS biosynthetic pathway, such that Lipid IV_{A} alone is transported to the outer membrane. In some other embodiments, the viable Gram-negative bacterial host cell comprises mutations wherein the mutations prevent association between KDO and Lipid IV_{A} in the LPS biosynthetic pathway, such that LipidA alone is transported to the outer membrane. In some embodiments, one or more mutations in KDO synthesis genes, or one or more mutations in the LPS biosynthetic pathway, include mutations in, but not limited to, any one or more of the genes encoding a protein chosen from a D-arabinose 5-phosphate isomerase, a KDO8P synthase, a CMP-KDO synthetase, a KDO8P phosphatase and/or a KDO transferase, an ATP-dependent translocator, an inner membrane protein (e.g., gutQ, kdsD, kdsA, kdsB, waaA, msbA, and yhjD), or any other biosynthetic, processing, or trafficking gene. In some embodiments, the cell lacks or substantially lacks synthesis of KDO proteins. In some embodiments, the outer membrane of the viable Gram-negative bacteria expresses lipid IV_{A}. In some embodiments, the Gram-negative bacterial host cell is E. coli. In certain embodiments, the disclosure provides a method of producing lipid IV_{A}, comprising extracting lipid IV_{A} from viable Gram-negative bacterial host cells.

In some embodiments, the disclosure provides viable Gram-negative bacteria (e.g., E. coli) lacking API expression as described herein.

In some embodiments, the disclosure provides a viable non-toxic (e.g., endotoxin free) Gram-negative bacterial cell (e.g., E. coli). The disclosure is not limited to a particular method of providing viable non-toxic Gram-negative bacterial cells. In some embodiments, viable non-toxic Gram-negative bacteria are provided through suppression of LPS expression in the outer membrane. The disclosure is not limited to a particular method of suppressing LPS expression in the outer membrane. In some embodiments, LPS expression is suppressed through suppression of API protein expression. The disclosure is not limited to a particular method of suppressing API expression. In some embodiments, API expression is suppressed through suppression of KDO protein expression. The disclosure is not limited to a particular method of suppressing KDO protein expression. In some embodiments, KDO protein expression is suppressed through, for example, mutation of the gutQ gene and the kdsD gene. In some embodiments, KDO protein expression at the outer membrane does not occur due to the KDO protein not associating with Lipid IV_{A}, such that only Lipid IV_{A} is transported to the outer membrane. For example, mutations in gutQ, kdsD, kdsA, kdsB, waaA msbA, and/or yhjD genes or mutations of any other biosynthetic, processing, or trafficking genes eliminate the formation of or membrane presentation of the KDO₂-Lipid IV_{A} complex, resulting in, for example, only the Lipid IV_{A} molecule being transported to the outer membrane and no subsequent LPS formation.

In a further preferred embodiment of the disclosure, a host cell as described herein, is further transformed with one or more genes of interest operably linked to a promoter and/or UTR. The gene of interest is on a plasmid or chromosome and is expressed in the host cell. In a specific embodiment, the host cell with a mutated outer membrane biosynthetic pathway leading to disruption of the pathway and comprising a mutation in the expression or the coding sequence of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase, is further transformed with one or more genes of interest operably linked to a promoter and/or UTR.

In some examples, the viable bacterial host cell as described herein can be used for production of a composition, preferably a vaccine, that stimulates the immune response. In other examples the outer membrane of the bacterial host cell can be used for the production of a composition or a vaccine, preferably for stimulating the immune response.

In certain embodiments, the disclosure provides an outer membrane vaccine or other composition for inducing an immune response against Gram-negative bacteria, the compositions comprising an outer membrane of viable Gram-negative bacterial cells of the disclosure. Such compositions may be used to induce immune responses in research, drug-screening, and therapeutic settings.

For example, a less toxic vaccine is produced using the viable bacterial host cell as described herein. Current vaccines can cause side effects due to endotoxins present in the vaccine preparation. It is contemplated that utilizing the viable non-toxic bacterial host cells or portions thereof as described herein where no LPS (e.g., endotoxin) is presented on the outer membrane bypasses side effects caused by endotoxin containing vaccine preparations. The disclosure finds utility in any vaccine preparation or other composition where endotoxin contamination is typically found. The viable bacterial host cells of the disclosure are also contemplated to find utility as live attenuated vaccines due to their LPS deficiency phenotype. As such, the disclosure finds use in developing outer membrane vaccines and other compositions for inducing immune responses that are free of endotoxin contamination that can be administered to subjects for immunization and research purposes.

In certain embodiments, the disclosure provides an adjuvant comprising lipid IV_{A} isolated from Gram-negative bacterial cells.

In a further example, a composition is provided comprising a viable cell having a disruption in the KDO₂-LipidA biosynthetic pathway, wherein the cell further comprises a mutation in the expression or the coding sequences of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase. Preferably the mutation provides an increased activity of at least one of the proteins. Preferably, such increased activity of the protein is the result of an overexpression of the respective gene. Alternatively, the increased activity of the protein is provided by introducing and expression of any one or more of the genes encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase. More preferably, the disrupted KDO₂-LipidA biosynthetic pathway results from mutations such that the cell is substantially free of KDO. Alternatively or preferably, the cell comprises mutations, which eliminate association between KDO₂ and Lipid IV_{A}.

Another example provides for a composition for use in inducing an immune response in a subject, comprising the outer membrane of the bacterial host cell as described herein.

Another example provides for a composition for use in immunizing a subject at risk of acquiring a condition, comprising the outer membrane of the bacterial host cell as described herein, wherein the condition is:
i) septic shock, wherein the Gram-negative bacteria is E. coli; or
ii) enteric fevers, wherein the Gram-negative bacteria is a Salmonella spp.

A further example provides for a method for inducing an immune response in a subject comprising administering a composition as described herein to the subject such that the administration induces an immune response.

In certain embodiments, the disclosure provides a method of treating an endotoxin related disorder, comprising administering to a subject with an endotoxin related disorder a composition comprising lipid IV_{A} isolated from Gram-negative bacterial host cells according to the disclosure.

Other exemplary embodiments provide for a method of producing LipidIV_{A} comprising extracting LipidIV_{A} from the bacterial host cell as described herein or alternatively a method for producing LipidA comprising extracting LipidA from the bacterial host cell as described herein.

A further embodiment provides a method of screening for an anti-pyrogenic agent, comprising the use of a bacterial host cell as described herein.

In some embodiments, the viable bacterial host cell of the disclosure (e.g., E. coli) and comprising a disrupted outer membrane biosynthetic pathway is used for purposes of pharmaceutical screening (e.g., screening for anti-pyrogenic agents). Such host cell has a very low permeability barrier, making it particularly susceptible to large, hydrophobic drug molecules that normally cannot penetrate the outer membrane. This lowered permeability barrier makes the use of permeabilizing agents superfluous in whole cell bioassays of compound libraries, which would normally use permeabilizing agents such as toluene, EDTA, cationic peptides, etc., to help identify hits by facilitating penetration of the outer membrane.

In some embodiments, the viable bacterial host cell as described herein finds utility in generating therapeutic antibodies for therapeutic and research applications. For example, in some embodiments subjects are actively immunized using the viable bacterial host cells or portions thereof (e.g., membrane preparations), and antibodies prepared from human hyper-immune sera are then used to passively protect subjects against bacterial infection and sepsis. However, the generation of therapeutic antibodies is more traditionally accomplished in host animals such as, but are not limited to, primates, rabbits, dogs, guinea pigs, mice, rats, sheep, goats, etc. Therapeutic antibodies, for example, are created using the viable bacterial host cells as immunogens themselves for creating antibodies in host animals for administration to human subjects. The viable bacterial host cell as described herein additionally finds utility as hosts for presenting a foreign antigen (e.g., immunogenic peptide or protein) that is used to create therapeutic antibodies in a host animal. For example, the bacterial host cell, besides being substantially deficient in LPS, can be genetically manipulated (e.g., via established cloning methods known to those skilled in the art) to express non-native proteins and peptides that find use as immunogens for antibody production. Such immunogens include, but are not limited to, peptides for targeting antibodies to cancer cells and other disease-causing cells, viral coat proteins for viral cell targeting, and the like.

In some embodiments, the disclosure provides non-toxic viable bacterial host cells useful for presenting immunogenic proteins for therapeutic antibody production. An antibody against an immunogenic protein may be any monoclonal or polyclonal antibody, as long as it can recognize the antigenic protein. Antibodies can be produced according to a conventional antibody or antiserum preparation process known to those skilled in the art.

In some embodiments, the viable bacterial cells can be genetically engineered via cloning methods known to those skilled in the art (see Sambrook et al., Molecular Cloning; A Laboratory Manual, Cold Spring Harbor Laboratory Press) to express, produce and display non-native proteins and peptides such as, but not limited to, LPS from other bacterial organisms, unique lipid derivatives, human protein or peptide production, non-human protein or peptide production, vaccine production, bioproduct, metabolite, oligosaccharide, glycolipid, glycoprotein and the like. Such products produced find utility in a variety of applications, including but not limited to, (clinical) therapeutics, food, feed and basic research endeavours.

Further according to the disclosure, a method is provided for the production of a bioproduct or metabolite using a genetically modified host cell. The method comprising the steps of:
providing a host cell, which has been genetically modified, such, that at least the cell is able to produce the bioproduct or metabolite wherein the unmodified host cell is not able to produce the bioproduct or metabolite, due to the introduction of at least one heterologous gene, encoding the bioproduct or an intermediate thereof, which is expressed in the host cell;
cultivating and/or growing the genetically modified host cell in a cultivation medium enabling to production of the bioproduct or metabolite thereby producing the bioproduct or metabolite obtainable from the medium the host cell is cultivated in;

characterized in that the host cell is a bacterial host cell as described herein.

The bacterial host cell as described herein can thus be used for the production of a bioproduct or metabolite as described herein. Alternatively, such bacterial host cell can be used for the production of Lipid IV_{A} or Lipid A.

The disclosure is not limited to particular methods of constructing viable Gram-negative bacteria substantially lacking outer membrane LPS expression (e.g., through suppression of API expression; through mutation of the gutQ and/or kdsD genes; through suppression of KDO expression; through inhibiting associations between KDO and Lipid IV_{A}; through mutations of the kdsA, and/or kdsB, and/or waaA and/or msbA and/or yhjD genes, or other biosynthetic, processing, or trafficking genes; through suppression of lipid IV_{A} expression; through mutations of the lpxM gene, or other biosynthetic, processing, or trafficking genes for lipid IV_{A}) in combination with overexpression of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase.

The disclosure is not limited to specific culture conditions for the growth of bacterial host cells according to the disclosure (e.g., bacterial host cells with mutations as described herein, or other biosynthetic, processing, or trafficking genes). For illustrative purposes, bacterial host cells can be grown in any standard liquid medium suitable for bacterial growth, such as LB medium (Difco, Detroit Mich.), Nutrient broth (Difco), Tryptic Soy broth (Difco), or M9 minimal broth (Difco), using conventional culture techniques that are appropriate for the bacterial cell being grown (Miller, 1991, supra). As an alternative, the bacteria can be cultured on solid media such as L-agar (Difco), Nutrient agar (Difco), Tryptic Soy agar (Difco), or M9 minimal agar (Difco). For Gram-negative bacterial cells wherein the cell comprises the mutations kdsD and/or gutQ, an exogenous D-arabinose 5-phosphate source is generally used for bacterial growth and survival. Prior art teaches that overexpression of the msbA gene in cells comprising the kdsD and/or gutQ mutations is an alternative to supplementation by D-arabinose 5-phosphate for bacterial growth and survival.

The disclosure contemplates the use of any technique for introducing genetic mutations within bacteria. Such techniques include, but are not limited to, non-specific mutagenesis, using chemical agents such as N-methyl-N′-nitro-N-nitrosoguanidine, acridine orange, ethidium bromide, or non-lethal exposure to ultraviolet light. Alternatively, the mutations can be introduced using Tn10 mutagenesis, bacteriophage-mediated transduction, lambda phage-mediated allelic exchange, or conjugational transfer, or site-directed mutagenesis using recombinant DNA techniques or any other known recombinant DNA technique. Any method for introducing mutations may be used and the mutations can be introduced in conjunction with one or more additional mutations. For example, in some embodiments the disclosure provides viable Gram-negative bacteria with more than one mutation such as mutations in the gutQ, kdsD, kdsA, kdsB, waaA msbA, yhjD genes, or mutations in any other biosynthetic, processing, or trafficking gene.

In some embodiments, the disclosure provides bacterial host cells (e.g., E. coli) comprising an outer membrane expressing lipid IV_{A}. The disclosure is not limited to a particular method of providing viable bacterial host cells comprising an outer membrane expressing lipid IV_{A}. In some embodiments, viable bacterial host cells comprising an outer membrane expressing lipid IV_{A} is accomplished through suppression of API protein expression. In some other embodiments, the disclosure provides bacterial host cells (e.g., E. coli) comprising an outer membrane expressing lipidA. The disclosure is not limited to a particular method of providing viable bacterial host cells comprising an outer membrane expressing lipidA. In some embodiments, viable bacterial host cells comprising an outer membrane expressing lipidA is accomplished through suppression of API protein expression.

In some embodiments, LPS free viable bacterial host cells comprising an outer membrane expressing Lipid IV_{A} is accomplished by inhibiting the association between KDO and Lipid IV_{A}, such that only Lipid IV_{A} is transported to the outer membrane (e.g., without KDO). The disclosure is not limited to a particular method of inhibiting the association between KDO and Lipid IV_{A}. In some embodiments, the association of KDO and Lipid IV_{A} is inhibited by, for example, mutations in the gutQ, kdsD, kdsA, kdsB, waaA, msbA and/or yhjD genes, or mutations of any other biosynthetic, processing, or trafficking genes. In some embodiments, the disclosure provides lipid IV_{A} isolated from viable non-toxic Gram-negative bacteria (e.g., E. coli). Lipid IV_{A} is used, for example, in studying mammalian septic shock signaling pathways, and as a building block in the synthesis of LPS-type molecules. Current methods for isolating lipid IV_{A} involve traditional total organic synthesis, degradation of mature LPS, or purification from conditional mutants that elaborate a heterogeneous LPS layer that contains a fraction of the desired lipid IV_{A}. Drawbacks for such methods include low lipid IV_{A} yield and high amounts of labor. Isolation of lipid IV_{A} from the viable non-toxic Gram-negative bacteria of the disclosure represents a significant improvement over such methods due to the outer membrane presence of lipid IV_{A}.

In some embodiments, the disclosure provides outer membrane vesicles isolated from viable non-toxic Gram-negative bacteria (e.g., E. coli). Lipid IV_{A} is an antagonist of septic shock signaling pathways, and a viable approach to treating patients with acute sepsis is to block the signaling pathway involving LPS. In some embodiments, isolated outer membrane vesicles from viable Gram-negative bacteria comprising an outer membrane expressing lipid IV_{A} are used to treat, or prophylactically prevent, sepsis related disorders. Outer membrane vesicles prepared from the viable non-toxic Gram-negative bacteria of the disclosure (e.g., the ΔAPI cell) contain lipid IV_{A} as an LPS antagonist.

In some embodiments, outer membrane vesicles isolated from viable Gram-negative bacteria (e.g., E. coli) are used for purposes of improved outer membrane vesicles-based vaccines. Outer membrane vesicles-based vaccines are often “detoxified’ by stripping away the LPS by harsh chemical treatments. Stripping methods, however, have a deleterious effect on protein components of the outer membrane vesicles-based vaccine, which can be good candidates to target antibodies against, particularly of cloned outer membrane proteins from other Gram-negative pathogens. Detoxification would not be necessary with the ΔAPI mutant cell as hosts, providing an additional level of safety.

In some embodiments, the disclosure provides Gram-negative bacteria comprising an outer membrane with both lipid IV_{A} and LPS expression. Separating the toxicity of LPS from the immunostimulatory properties is a major challenge to develop LPS based adjuvants or LPS based vaccines. Since the block in the ΔAPI cell is early in the LPS pathway, enzymes from other bacteria (which modify LPS with phosphate groups, ethanolamine, L-4-deoxy arabinose, different acyl chain lengths, etc.) and mutated enzymes with altered activities can be used to generate an array of LPS molecules with unique biological activities inside the cell. Many methods for such genetic manipulations already exist in Escherichia coli. Further, mature LPS synthesis can be restored by inclusion of D-arabinose 5-phosphate in the growth media, allowing one to control and optimize the amount and ratio of LPS derivatives to mature LPS. Such LPS “blends” may achieve the desired balance between immunostimulatory activity while retaining acceptable low levels of potential toxicity.

In some embodiments, viable non-toxic Gram-negative bacteria are used as hosts for the production of endotoxin free therapeutic molecules. The disclosure is not limited to particular therapeutic molecules. Traditionally, the production of therapeutic molecules in Gram-negative bacteria, whether it be outer membrane vesicles for vaccines, LPS type molecules (such as monophosphoryl lipid A (MPLA)) to be used as adjuvants, recombinant pharmaceutical proteins, macromolecules, or DNA for mammalian cell transfection/gene therapy, is plagued by the presence of endotoxin from the bacterial host. Contamination of the therapeutic molecule with endotoxin is a concern, as the immunogenic potential of LPS is well documented. Current production strategies to alleviate endotoxin contamination include various purification techniques, such as the kits marketed for endotoxin free DNA plasmid purification, followed by assays to measure endotoxin levels. As the ΔAPI cell does not produce endotoxin, such purification steps are not required. As such, the viable non-toxic Gram-negative bacterial cells of the disclosure (e.g., the ΔAPI cell) provide improved methods of isolating endotoxin free therapeutic molecules (e.g., lipid IV_{A}). For example, the ΔAPI cell is contemplated to be a host for the production of commercially important therapeutic molecules in an endotoxin-free environment using the well-studied Gram-negative bacteria. Additionally, cells comprising a mutation in a gene encoding any one or more of a D-arabinose 5-phosphate isomerase, a KDO8P synthase, a CMP-KDO synthetase, a KDO8P phosphatase and/or a KDO transferase, an ATP-dependent translocator, an inner membrane protein (e.g., gutQ, kdsD, kdsA, kdsB, waaA msbA, yhjD genes), or mutations in any other biosynthetic, processing, or trafficking bacterial genes are contemplated to be hosts for the production of commercially important therapeutic molecules in an endotoxin-free environment using Gram-negative bacteria.

Moreover, the disclosure relates to the following specific embodiments:

1. A viable bacterial host cell that can grow and divide with mutated outer membrane biosynthetic pathway leading to disruption of the pathway, characterized in that the cell further comprises a mutation in the expression or the coding sequence of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase.

2. Host cell according to embodiment 1, wherein the outer membrane biosynthetic pathway is the KDO₂-LipidA biosynthetic pathway.

3. Host cell according to any one of the embodiments 1 or 2, wherein the cell lacks i) KDO in outer membrane and/or ii) LPS expression.

4. Host cell according to any one of embodiment 1 to 3, wherein the bacterial mutant host cell displays LipidA in outer membrane.

5. Host cell according to any one of embodiment 1 to 4, wherein the disruption in the outer membrane biosynthetic pathway is caused by at least one mutation in a gene encoding a protein selected from the group consisting of D-arabinose 5-phosphate isomerase, KDO8P synthase, CMP-KDO synthetase, KDO8P phosphatase and/or KDO transferase.

6. Host cell according to any one of embodiment 1 to 5, wherein the disruption in the outer membrane biosynthetic pathway is caused by at least one mutation in a gene or expression of a gene encoding a protein selected from the group consisting of gutQ, kdsD, kdsA, kdsB, kdsC and waaA, wherein gutQ and kdsD encode a D-arabinose 5-phosphate isomerase, kdsA encodes a KDO8P synthase, kdsB encodes a CMP-KDO synthetase, kdsC encodes a KDO8P phosphatase and waaA encodes a KDO transferase.

7. Host cell according to any one of embodiment 1 to 3, or 5 to 6, wherein the bacterial mutant cell displays LipidIV_{A} in outer membrane and wherein the cell is additionally mutated in at least one gene or the expression of at least one gene encoding a protein selected from the group consisting of lauroyl acyltransferase and myristoyl-acyl carrier protein-dependent acyltransferase.

8. Host cell according to any one of embodiment 1 to 3, or 5 to 7, wherein the bacterial cell is additionally mutated in at least one gene or the expression of at least one gene encoding a protein selected from the group consisting of LpxL and/or LpxM.

9. Host cell according to any one of embodiment 1 to 8, wherein poly-β-1,6-N-acetyl-D-glucosamine outer membrane porin is encoded by pgaA, poly-β-1,6-N-acetyl-D-glucosamine N-deacetylase is encoded by pgaB, and/or poly-N-acetyl-D-glucosamine synthase activity is encoded by pgaD and pgaC.

10. Host cell according to any one of embodiment 1 to 9, wherein the mutation in the expression or the coding sequence of any one or more of the genes, encoding for the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase provides poly-N-acetylglucosamine overproduction.

11. Host cell according to any one of embodiment 1 to 10, wherein at least two of the genes encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase, are clustered in an operon.

12. Host cell according to any one of embodiment 1 to 11, wherein the mutation in the expression or the coding sequences of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase provides an increased activity of at least one of the proteins, preferably an overexpression of at least one, two, three or four of the proteins, more preferably an overexpression of the operon encoding the proteins.

13. Host cell according to any one of embodiment 1 to 12 wherein the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is an overexpression in the pgaABCD cluster.

14. Host cell according to any one of embodiment 1 to 13 wherein the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is provided by overexpressing or introducing and expressing, preferably overexpressing, any one or more genes encoding for the proteins selected from a Na⁺/H⁺ antiporter regulator and a sensor histidine kinase.

15. Host cell according to embodiment 14, wherein the Na⁺/H⁺ antiporter Regulator is a nhaR protein, preferably from E. coli and/or sensor histidine kinase is a resC protein, preferably from E. coli.

16. Host cell according to any one of embodiment 1 to 15 wherein the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is provided a reduced or abolished expression of a carbon storage regulator, preferably the carbon storage regulator is a csrA, preferably from E. coli.

17. Host cell according to any one of embodiment 1 to 16, comprising a mutation, which eliminates association between KDO₂ and LipidIV_{A}.

18. Host cell according to any one of embodiment 1 to 17, wherein the disruption in the outer membrane biosynthetic pathway is caused by at least one mutation in the gene encoding a KDO transferase.

19. Host cell according to any one of embodiment 1 to 18, wherein the disruption in the outer membrane biosynthetic pathway results in a lack of expression of D-arabinose 5-phosphate isomerase (API).

20. Host cell according to any one of embodiment 1 to 19, wherein the host cell further comprises a mutation in the expression or the coding sequence of a gene selected from an ATP-dependent translocator, inner membrane protein, D-arabinose 5-phosphate isomerase, Palmitoleoyl acyltransferase, Lipid A palmitoyltransferase, and/or phosphoethanolamine transferase, preferably the ATP-dependent translocator is an ATP-dependent Lipid A-core flippase; preferably the inner membrane protein is a transmembrane transporter.

21. Host cell according to any one of embodiment 1 to 20, wherein the host cell further comprises a mutation in the expression or the coding sequence of a gene selected from msbA, yhjD, gutQ, kdsD, lpxP, pagP, eptA.

22. Host cell according to any one of embodiment 1 to 21, wherein the host cell is a Gram-negative bacterial cell.

23. Host cell according to any one of embodiment 1 to 22, wherein the host cell is selected from the group consisting of Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Mycobacteria spp., Citrobacter spp., Chlamydia spp., Brucella spp., Pseudomonas spp., Helicobacter spp., Moraxella spp., Stenotrophomonas spp., Bdellovibrio spp., Acinetobacter spp., Enterobacter spp. and Vibrio spp.

24. Host cell according to any one of embodiment 1 to 23, wherein the host cell is selected from Escherichia spp., Salmonella spp., and Pseudomonas spp.

25. Host cell according to any one of embodiment 1 to 24, wherein the host cell is E. coli.

26. Host cell according to any one of embodiment 1 to 25, wherein the host cell is E. coli selected from the group consisting of K-12 strain, W3110, MG1655, B/r, BL21, O157:h7, 042, 101-1, 1180, 1357, 1412, 1520, 1827-70, 2362-75, 3431, 53638, 83972, 929-78, 98NK2, ABU 83972, B, B088, B171, B185, B354, B646, B7A, C, c7122, CFT073, DH1, DH5a, E110019, E128010, E74/68, E851/71, EAEC 042, EPECa11, EPECa12, EPECa14, ETEC, H10407, F11, F18+, FVEC1302, FVEC1412, GEMS_EPEC1, HB101, HT115, KO11, LF82, LT-41, LT-62, LT-68, MS107-1, MS119-7, MS124-1, MS 145-7, MS 79-2, MS 85-1, NCTC 86, Nissle 1917, NT:H19, NT:H40, NU14, O103:H2, O103:HNM, O103:K+, O104:H12, O108:H25, O109:H9, O111H−, O111:H19, O111:H2, O111:H21, O11:NM, O115:H−, O115:HMN, O115:K+, O119:H6, O119:UT, O124:H40, O127a:H6, O127:H6, O128:H2, O131:H25, O136:H−, O139:H28 (strain E24377A/ETEC), O13:H11, O142:H6, O145:H−, O153:H21, O153:H7, O154:H9, O157:12, O157:H−, O157:H12, O157:H43, O157:H45, O157:H7 EDL933, O157:NM, O15:NM, O177:H11, O17:K52:H18 (strain UMN026/ExPEC), O180:H−, O1:K1/APEC, O26, O26:H−, O26:H11, O26:H11:K60, O26:NM, O41:H−, O45:K1 (strain S88/ExPEC), O51:H−, O55:H51, O55:H6, O55:H7, O5:H−, O6, O63:H6, O63:HNM, O6:K15:H31 (strain 536/UPEC), O7:K1 (strain IAI39/ExPEC), O8 (strain IAI1), O81 (strain ED1a), O84:H−, O86a:H34, O86a:H40, O90:H8, O91:H21, O9:H4 (strain HS), O9:H51, ONT:H−, ONT:H25, OP50, Orough:H12, Orough:H19, Orough:H34, Orough:H37, Orough:H9, OUT:H12, OUT:H45, OUT:H6, OUT:H7, OUT:HNM, OUT:NM, RN587/1, RS218, 55989/EAEC, B/BL21, B/BL21-DE3, SE11, SMS-3-5/SECEC, UTI89/UPEC, TA004, TA155, TX1999, and Vir68.

27. Host cell according to any one of embodiment 1 to 26, wherein the cell is further transformed with one or more genes of interest operably linked to a promoter and/or UTR.

28. Host cell according to embodiment 27, wherein the gene of interest is on a plasmid or chromosome and is expressed in the host cell.

29. Host cell according to any one of embodiment 1 to 28, wherein the poly-β-1,6-N-acetyl-D-glucosamine outer membrane porin is preferably a polypeptide sequence comprising a sequence having 80% or more sequence identity to the sequence as given by SEQ ID NO:7, poly-β-1,6-N-acetyl-D-glucosamine N-deacetylase is preferably a polypeptide sequence comprising a sequence having 80% or more sequence identity to the sequence as given by SEQ ID NO:8, poly-N-acetyl-D-glucosamine synthase is preferably a polypeptide sequence comprising a sequence having 80% or more sequence identity to the sequence as given by any one of SEQ ID NO:9 or SEQ ID NO:10, the ATP-dependent translocator is preferably a polypeptide sequence comprising a sequence having 80% or more sequence identity to the sequence as given by SEQ ID NO:16; the inner membrane protein is preferably a polypeptide sequence comprising a sequence having 80% or more sequence identity to the sequence as given by SEQ ID NO:17, lauroyl acyltransferase is preferably a polypeptide sequence comprising a sequence having 80% or more sequence identity to the sequence as given by SEQ ID NO:11; D-arabinose 5-phosphate isomerase is preferably a polypeptide sequence comprising a sequence having 80% or more sequence identity to the sequence as given by any one of SEQ ID NO:2 or SEQ ID NO:1, Palmitoleoyl acyltransferase is preferably a polypeptide sequence comprising a sequence having 80% or more sequence identity to the sequence as given by SEQ ID NO:18, Lipid A palmitoyltransferase is preferably a polypeptide sequence comprising a sequence having 80% or more sequence identity to the sequence as given by SEQ ID NO:19, phosphoethanolamine transferase is preferably a polypeptide sequence comprising a sequence having 80% or more sequence identity to the sequence as given by SEQ ID NO:20, myristoyl-acyl carrier protein-dependent acyltransferase is preferably a polypeptide sequence comprising a sequence having 80% or more sequence identity to the sequence as given by SEQ ID NO:12, KDO8P synthase is preferably a polypeptide sequence comprising a sequence having 80% or more sequence identity to the sequence as given by SEQ ID NO:3, CMP-KDO synthetase is preferably a polypeptide sequence comprising a sequence having 80% or more sequence identity to the sequence as given by SEQ ID NO:4, KDO transferase is a polypeptide sequence comprising a sequence having 80% or more sequence identity to the sequence as given by SEQ ID NO:6, KDO8P phosphatase is preferably a polypeptide sequence comprising a sequence having 80% or more sequence identity to the sequence as given by SEQ ID NO:5, Na⁺/H⁺ antiporter regulator is preferably a polypeptide sequence comprising a sequence having 80% or more sequence identity to the sequence as given by SEQ ID NO:13, the sensor histidine kinase is preferably a polypeptide sequence comprising a sequence having 80% or more sequence identity to the sequence as given by SEQ ID NO:14, and/or the carbon storage regulator is preferably a polypeptide sequence comprising a sequence having 80% or more sequence identity to the sequence as given by SEQ ID NO:15.

30. Isolated host cell according to any one of embodiments 1 to 29.

31. A composition comprising an outer membrane of the bacterial host cell of any one of embodiment 1 to 30.

32. A composition comprising the host cell of any one of embodiment 1 to 30.

33. A composition for use in inducing an immune response in a subject, comprising the outer membrane of the bacterial host cell of any one of embodiment 1 to 30.

34. A composition for use in immunizing a subject at risk of acquiring a condition, comprising the outer membrane of the bacterial host cell of any one of embodiment 1 to 30, wherein the condition is:
i) septic shock, wherein the Gram-negative bacteria is E. coli; or
ii) enteric fevers, wherein the Gram-negative bacteria is a Salmonella spp.

35. A method for inducing an immune response in a subject comprising administering a composition of any one of embodiment 31 to 34 to the subject such that the administration induces an immune response.

36. A method of producing LipidIV_{A} comprising extracting LipidIV_{A} from the bacterial host cell of any one of embodiment 7 to 30.

37. A method for producing LipidA comprising extracting LipidA from the bacterial host cell of any one of embodiment 1 to 6, or 9 to 30.

38. A method of screening for an anti-pyrogenic agent, comprising the use of a bacterial host cell according to embodiment 1 to 30.

39. A method for the production of a bioproduct or metabolite using a genetically modified host cell, the method comprising the steps of:
providing a host cell, which has been genetically modified, such, that at least the cell is able to produce the bioproduct or metabolite wherein the unmodified host cell is not able to produce the bioproduct or metabolite, due to the introduction of at least one heterologous gene, encoding the bioproduct or an intermediate thereof, which is expressed in the host cell;
cultivating and/or growing the genetically modified host cell in a cultivation medium enabling production of the bioproduct or metabolite thereby producing the bioproduct or metabolite obtainable from the medium the host cell is cultivated in;

characterized in that the host cell is a bacterial host cell according to any one of embodiment 1 to 30.

40. Use of the bacterial host cell of any one of embodiment 1 to 30 for the production of a bioproduct or metabolite.

41. Use of a host cell according to any one of embodiment 1 to 30 for the production of Lipid IV_{A} or Lipid A.

42. Use of a host cell according to any one of embodiment 1 to 30 for immunizing a subject.

43. An isolated viable bacterial host cell, wherein the cell comprises a first mutation leading to a disruption of the KDO₂-LipidA biosynthetic pathway, and a second mutation in the expression or the coding sequence of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase, and wherein the bacterial cell lacks KDO and displays LipidA in its outer membrane.

44. An isolated viable bacterial host cell, wherein the cell comprises a first mutation leading to a disruption of the KDO₂-LipidA biosynthetic pathway, and a second mutation in the expression or the coding sequence of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase, and wherein the bacterial cell lacks KDO and displays LipidIV_{A} in its outer membrane.

45. The bacterial cell of embodiment 43, wherein the first mutation resides in a gene selected from the group consisting of D-arabinose 5-phosphate isomerase, KDO8P synthase, CMP-KDO synthetase, KDO8P phosphatase and KDO transferase.

46. The bacterial cell of embodiment 44, wherein the first mutation resides in a gene selected from the group consisting of D-arabinose 5-phosphate isomerase, KDO8P synthase, CMP-KDO synthetase, KDO8P phosphatase and KDO transferase and wherein the cell further comprises at least one mutation in a gene selected from the group consisting of lauroyl acyltransferase and myristoyl-acyl carrier protein-dependent acyltransferase.

47. The bacterial host of any one of embodiment 43 to 46, wherein the second mutation provides poly-N-acetylglucosamine overproduction.

48. A bacterial cell according to embodiment 47, wherein the second mutation is an overexpression.

49. The bacterial cell of any one of embodiment 43 to 48, wherein the first mutation results in a lack of expression of D-arabinose 5-phosphate isomerase (API).

50. The bacterial cell of any one of embodiment 43 or 48, wherein the first mutation results in a lack of expression of KDO transferase.

51. The bacterial cell of any one of embodiment 43 to 46, wherein the cell further comprises a mutation in the expression or the coding sequence of any one or more of the genes selected from ATP-dependent translocator, inner membrane protein, D-arabinose 5-phosphate isomerase, Palmitoleoyl acyltransferase, Lipid A palmitoyltransferase, and/or phosphoethanolamine transferase; preferably the ATP-dependent translocator is an ATP-dependent Lipid A-core flippase; preferably the inner membrane protein is a transmembrane transporter.

52. The bacterial cell of any one of embodiment 43 to 51, wherein the cell is selected from the group consisting of Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Mycobacterium spp., Citrobacter spp., Chlamydia spp., Brucella spp., Pseudomonas spp., Helicobacter spp, Moraxella spp., Stenotrophomonas spp., Bdellovibrio spp., Acinetobacter spp., Enterobacter spp. and Vibrio spp.

53. The bacterial cell of any one of embodiment 43 to 52, wherein the cell is selected from the group consisting of Escherichia spp., Salmonella spp., and Pseudomonas spp.

54. The bacterial cell of embodiment 53, wherein the cell is E. coli.

55. The bacterial cell of any one of embodiment 43 to 54, wherein the cell is transformed with one or more genes of interest operably linked to a promoter and/or UTR.

56. The bacterial cell of embodiment 55, wherein the gene of interest is on a plasmid or chromosome and is expressed in the bacterial cell.

57. A composition comprising an outer membrane of the viable Escherichia coli cell of embodiment 54.

58. A composition, comprising the viable Escherichia coli cell of embodiment 54.

59. A composition comprising the viable bacterial cell of any one of embodiment 43 to 56.

60. The composition of any one of embodiment 57 to 59, wherein the viable bacterial cell lacks D-arabinose 5-phosphate isomerase (API) expression.

61. A method of producing Lipid IV_{A}, comprising extracting Lipid IV_{A} from the viable bacterial cell of any one of embodiment 44 to 56.

62. A method of producing LipidA, comprising extracting LipidA from the viable Gram-negative bacterial cell of any one of embodiment 43, 45 to 56.

63. A composition comprising viable cell having a disruption in the KDO-LipidA biosynthetic pathway, wherein the cell further comprises a mutation in the expression or the coding sequences of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase, preferably the mutation provides an increased activity of at least one of the proteins.

64. The composition of embodiment 63, wherein the viable bacterial cell comprises mutations such that the cell is substantially free of KDO.

65. The composition of any one of embodiment 63 or 64, wherein the viable bacterial cell comprises mutations such that the mutations eliminate association between KDO₂ and Lipid IV_{A}.

66. The composition of any one of embodiment 63 to 65, wherein the cell further comprises a mutation in the expression or the coding sequence of any one or more of a gene selected from an ATP-dependent translocator, inner membrane protein, D-arabinose 5-phosphate isomerase, Palmitoleoyl acyltransferase, Lipid A palmitoyltransferase, and/or phosphoethanolamine transferase, preferably the ATP-dependent translocator is an ATP-dependent Lipid A-core flippase; preferably the inner membrane protein is a transmembrane transporter.

67. The composition of any one of embodiment 63 to 66, wherein the outer membrane of the viable bacterial cell expresses LipidA.

68. A composition for inducing an immune response comprising a composition of any one of embodiment 63 to 67.

69. The composition of any one of embodiment 63 to 68, wherein the bacterial cell further overexpresses an ATP-dependent translocator, preferably an ATP-dependent Lipid A-core flippase.

70. The composition of any one of embodiment 63 to 69, wherein the outer membrane is substantially free of LPS expression.

71. A method for inducing an immune response in a subject comprising administering a composition of any one of embodiment 63 to 70 to the subject such that the administration induces an immune response.

Moreover, the disclosure relates to the following preferred specific embodiments:

1. A viable bacterial host cell with mutated KDO₂-LipidA biosynthetic pathway leading to disruption of the pathway, characterized in that the cell further comprises a mutation in the expression or the coding sequence of any one or more of a gene encoding for the proteins selected from the group of poly-β-1,6-N-acetyl-D-glucosamine outer membrane porin, poly-β-1,6-N-acetyl-D-glucosamine N-deacetylase, and/or poly-N-acetyl-D-glucosamine synthase.

2. Host cell according to preferred embodiment 1, wherein the cell lacks KDO in outer membrane.

3. Host cell according to preferred embodiment 1, wherein the cell is substantially free of LPS expression.

4. Host cell according to any one of preferred embodiment 1 to 3, wherein the bacterial mutant host cell displays LipidA in outer membrane.

5. Host cell according to any one of preferred embodiment 1 to 4, wherein the disruption in the outer membrane biosynthetic pathway is caused by at least one mutation in a gene encoding a protein selected from the group consisting of D-arabinose 5-phosphate isomerase, KDO8P synthase, CMP-KDO synthetase, KDO8P phosphatase and KDO transferase.

6. Host cell according to any one of preferred embodiment 1 to 5, wherein the disruption in the outer membrane biosynthetic pathway is caused by at least one mutation in a gene or expression of a gene encoding a protein selected from the group consisting of gutQ, kdsD, kdsA, kdsB, kdsC and waaA, wherein gutQ and kdsD encode a D-arabinose 5-phosphate isomerase, kdsA encodes a KDO8P synthase, kdsB encodes a CMP-KDO synthetase, kdsC encodes a KDO8P phosphatase and waaA encodes a KDO transferase.

7. Host cell according to any one of preferred embodiment 1 to 6, wherein the bacterial mutant cell displays LipidIV_{A} in outer membrane and wherein the cell is additionally mutated in at least one gene or the expression of at least one gene encoding for protein selected from the group consisting of lauroyl acyltransferase and myristoyl-acyl carrier protein-dependent acyltransferase.

8. Host cell according to any one of preferred embodiment 1 to 7, wherein the bacterial cell is additionally mutated in at least one gene or the expression of at least one gene encoding a protein selected from the group consisting of LpxL and/or LpxM.

9. Host cell according to any one of preferred embodiment 1 to 8, wherein poly-β-1,6-N-acetyl-D-glucosamine outer membrane porin is encoded by pgaA, poly-β-1,6-N-acetyl-D-glucosamine N-deacetylase is encoded by pgaB, and/or poly-N-acetyl-D-glucosamine synthase activity is encoded by pgaD and pgaC.

10. Host cell according to any one of preferred embodiments 1 to 9, wherein the mutation in the expression or the coding sequence of any one or more of the genes, encoding for the proteins selected from a poly-β-1,6-N-acetyl-D-glucosamine outer membrane porin, a poly-β-1,6-N-acetyl-D-glucosamine N-deacetylase, and/or a poly-N-acetyl-D-glucosamine synthase provides poly-N-acetylglucosamine overproduction.

11. Host cell according to any one of preferred embodiments 1 to 10, wherein at least two of the genes encoding for the proteins selected from an poly-β-1,6-N-acetyl-D-glucosamine outer membrane porin, poly-β-1,6-N-acetyl-D-glucosamine N-deacetylase, and/or poly-N-acetyl-D-glucosamine synthase, are clustered in an operon.

12. Host cell according to any one of preferred embodiments 1 to 11, wherein the mutation in the expression or the coding sequences of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase provides an increased activity of at least one of the proteins, preferably an overexpression of at least one, two, three or four of the proteins, more preferably an overexpression of the operon encoding the proteins.

13. Host cell according to any one of preferred embodiment 1 to 12 wherein the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is an overexpression in the pgaABCD cluster.

14. Host cell according to any one of preferred embodiment 1 to 13, wherein the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is provided by overexpressing or introducing and expressing, preferably overexpressing, any one or more genes encoding for the proteins selected from a Na⁺/H⁺ antiporter regulator and a sensor histidine kinase.

15. Host cell according to preferred embodiment 14, wherein the Na⁺/H⁺ antiporter regulator is a nhaR protein, preferably from E. coli and/or the sensor histidine kinase is a resC protein, preferably from E. coli.

16. Host cell according to any one of preferred embodiment 1 to 15, wherein the mutation in the expression or the coding sequence of any one or more of the genes, encoding for the proteins selected from a poly-β-1,6-N-acetyl-D-glucosamine outer membrane porin, poly-β-1,6-N-acetyl-D-glucosamine N-deacetylase, and/or poly-N-acetyl-D-glucosamine synthase is provided a reduced or abolished expression of a carbon storage regulator, preferably the carbon storage regulator is a csrA, preferably from E. coli.

17. Host cell according to any one of preferred embodiment 1 to 16, comprising a mutation, which eliminates association between KDO₂ and LipidIV_{A}.

18. Host cell according to any one of preferred embodiment 1 to 17, wherein the disruption in the outer membrane biosynthetic pathway is caused by at least one mutation in the gene encoding a KDO transferase.

19. Host cell according to any one of preferred embodiments 1 to 18, wherein the disruption in the outer membrane biosynthetic pathway results in a lack of expression of D-arabinose 5-phosphate isomerase (API).

20. Host cell according to any one of preferred embodiments 1 to 19, wherein the host cell further comprises a mutation in the expression or the coding sequence of a gene selected from an ATP-dependent translocator, inner membrane protein, D-arabinose 5-phosphate isomerase, Palmitoleoyl acyltransferase, Lipid A palmitoyltransferase, and/or phosphoethanolamine transferase, preferably the ATP-dependent translocator is an ATP-dependent Lipid A-core flippase; preferably the inner membrane protein is a transmembrane transporter.

21. Host cell according to any one of preferred embodiments 1 to 20, wherein the host cell further comprises a mutation in the expression or the coding sequence of a gene selected from msbA, yhjD, gutQ, kdsD, lpxP, pagP, eptA.

22. Host cell according to any one of preferred embodiments 1 to 21, wherein the host cell is a Gram-negative bacterial cell.

23. Host cell according to any one of preferred embodiments 1 to 22, wherein the host cell is selected from the group consisting of Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Mycobacteria spp., Citrobacter spp., Chlamydia spp., Brucella spp., Pseudomonas spp., Helicobacter spp. and Vibrio spp., preferably selected from Escherichia spp., Salmonella spp., and Pseudomonas spp., most preferably the host cell is E. coli.

24. Host cell according to any one of preferred embodiments 1 to 23, wherein the cell is further transformed with one or more genes of interest operably linked to a promoter and/or UTR.

25. Host cell according to preferred embodiment 24, wherein the gene of interest is on a plasmid or chromosome and is expressed in the host cell.

26. Host cell according to any one of preferred embodiment 1 to 25, wherein the poly-β-1,6-N-acetyl-D-glucosamine outer membrane porin is preferably a polypeptide sequence comprising a sequence having 80% or more sequence identity to the sequence as given by SEQ ID NO:7 and having poly-β-1,6-N-acetyl-D-glucosamine transmembrane transporter activity, poly-β-1,6-N-acetyl-D-glucosamine N-deacetylase is preferably a polypeptide sequence comprising a sequence having 80% or more sequence identity to the sequence as given by SEQ ID NO:8 and having poly-β-1,6-N-acetyl-D-glucosamine N-deacetylase activity, poly-N-acetyl-D-glucosamine synthase is preferably a polypeptide sequence comprising a sequence having 80% or more sequence identity to the sequence as given by any one of SEQ ID NO:9 or SEQ ID NO:10 and having poly-N-acetyl-D-glucosamine synthase activity, the ATP-dependent translocator is preferably a polypeptide sequence comprising a sequence having 80% or more sequence identity to the sequence as given by SEQ ID NO:16 and having ATP-dependent translocator activity; the inner membrane protein is preferably a polypeptide sequence comprising a sequence having 80% or more sequence identity to the sequence as given by SEQ ID NO:17 and having transmembrane transporter activity, lauroyl acyltransferase is preferably a polypeptide sequence comprising a sequence having 80% or more sequence identity to the sequence as given by SEQ ID NO:11 and having lauroyl acyltransferase activity; D-arabinose 5-phosphate isomerase is preferably a polypeptide sequence comprising a sequence having 80% or more sequence identity to the sequence as given by any one of SEQ ID NO:2 or SEQ ID NO:1 and having D-arabinose 5-phosphate isomerase activity, Palmitoleoyl acyltransferase is preferably a polypeptide sequence comprising a sequence having 80% or more sequence identity to the sequence as given by SEQ ID NO:18 and having Palmitoleoyl acyltransferase activity, Lipid A palmitoyltransferase is preferably a polypeptide sequence comprising a sequence having 80% or more sequence identity to the sequence as given by SEQ ID NO:19 and having Lipid A palmitoyltransferase activity, phosphoethanolamine transferase is preferably a polypeptide sequence comprising a sequence having 80% or more sequence identity to the sequence as given by SEQ ID NO:20 and having phosphoethanolamine transferase activity, myristoyl-acyl carrier protein-dependent acyltransferase is preferably a polypeptide sequence comprising a sequence having 80% or more sequence identity to the sequence as given by SEQ ID NO:12 and having myristoyl-acyl carrier protein-dependent acyltransferase activity, KDO8P synthase is preferably a polypeptide sequence comprising a sequence having 80% or more sequence identity to the sequence as given by SEQ ID NO:3 and having KDO8P synthase activity, CMP-KDO synthetase is preferably a polypeptide sequence comprising a sequence having 80% or more sequence identity to the sequence as given by SEQ ID NO:4 and having CMP-KDO synthetase activity, KDO transferase is a polypeptide sequence comprising a sequence having 80% or more sequence identity to the sequence as given by SEQ ID NO:6 and having KDO transferase activity, KDO8P phosphatase is preferably a polypeptide sequence comprising a sequence having 80% or more sequence identity to the sequence as given by SEQ ID NO:5 and having KDO8P phosphatase activity, Na⁺/H⁺ antiporter regulator is preferably a polypeptide sequence comprising a sequence having 80% or more sequence identity to the sequence as given by SEQ ID NO:13 and having Na⁺/H⁺ antiporter regulator activity, the sensor histidine kinase is preferably a polypeptide sequence comprising a sequence having 80% or more sequence identity to the sequence as given by SEQ ID NO:14 and having sensor histidine kinase activity, and/or the carbon storage regulator is preferably a polypeptide sequence comprising a sequence having 80% or more sequence identity to the sequence as given by SEQ ID NO:15 and having carbon storage regulator activity.

27. Isolated host cell according to any one of preferred embodiment 1 to 26.

28. A composition comprising an outer membrane of the bacterial host cell of any one of preferred embodiments 1 to 27.

29. A composition comprising the host cell of any one of preferred embodiments 1 to 27.

30. A composition for use in inducing an immune response in a subject, comprising the outer membrane isolated from the bacterial host cell of any one of preferred embodiments 1 to 27.

31. A composition for use in immunizing a subject at risk of acquiring a condition, comprising the outer membrane of the bacterial host cell of any one of preferred embodiments 1 to 27, wherein the condition is:
i) septic shock, wherein the Gram-negative bacteria is E. coli; or
ii) enteric fevers, wherein the Gram-negative bacteria is a Salmonella spp.

32. A composition according to any one of preferred embodiments 28 to 31 for use in a method comprising administering the composition to a subject such that the administration induces an immune response in the subject.

33. A method for inducing an immune response in a subject comprising administering a composition of any one of preferred embodiments 28 to 31 to the subject such that the administration induces an immune response in the subject.

34. A method of producing LipidIV_{A} comprising extracting LipidIV_{A} from the bacterial host cell of any one of preferred embodiments 7 to 27.

35. A method for producing LipidA comprising extracting LipidA from the bacterial host cell of any one of preferred embodiments 1 to 6, or 9 to 27.

36. A method of screening for an anti-pyrogenic agent, comprising the use of a bacterial host cell according to preferred embodiments 1 to 27.

37. A method for the production of a bioproduct or metabolite using a genetically modified host cell, the method comprising the steps of:
providing a host cell, which has been genetically modified, such, that at least the cell is able to produce the bioproduct or metabolite wherein the unmodified host cell is not able to produce the bioproduct or metabolite, due to the introduction of at least one heterologous gene, encoding the bioproduct or an intermediate thereof, which is expressed in the host cell;
cultivating and/or growing the genetically modified host cell in a cultivation medium enabling production of the bioproduct or metabolite thereby producing the bioproduct or metabolite obtainable from the medium the host cell is cultivated in;

characterized in that the host cell is a bacterial host cell according to any one of preferred embodiments 1 to 27.

38. Use of the bacterial host cell of any one of preferred embodiments 1 to 27 for the production of a bioproduct or metabolite.

39. Use of a host cell according to any one of preferred embodiments 1 to 27 for the production of Lipid IV_{A} or Lipid A.

40. A host cell according to any one of preferred embodiments 1 to 27 for use in an immunization method of a subject.

The following examples will serve as further clarification of the disclosure and are not intended to be limiting._{[JLS1]}

### EXAMPLES

### Example 1: Materials and Methods for Escherichia coli

Chemicals

Tryptone and yeast extract were procured from Becton Dickinson. All other chemicals were purchased from Sigma-Aldrich, unless stated otherwise.

Oligonucleotides and Molecular Reagents

Oligonucleotides were purchased from Integrated DNA Technologies (Leuven, Belgium). Sequencing services were conducted by LGC genomics (Berlin, Germany). Deoxynucleotides, agarose and ethidium bromide were purchased from Thermo Fisher Scientific. QIAprep Spin Miniprep kit was used for plasmid isolation (QIAgen, USA). Analytik Jena kits were used for PCR purification (Jena, Germany).

Strains

E. coli K-12 MG1655 ΔthyA was used as abase strain for all future modifications. E. coli One Shot Top10 Electrocomp™ cells (Life Technologies) were solely used for cloning purposes. An overview of the strains used is shown in Table 1. E. coli K-12 MG1655 was obtained from the Coli Genetic Stock Center (US), CGSC Strain #: 7740, in March 2007.

TABLE 1

| | |
|---|---|
| sNS5.01 | E. coli K-12 MG1655 ΔthyA::sl |
| sNS5.02 | sNS5.01 + pGEM-P14-pga ΔwaaA::thyA |
| sNS5.03 | sNS5.01 ΔsibD_siBE::attL_P14-pga-attR |

Media and Culture Conditions

The culture medium lysogeny broth (LB) consisting of 1% tryptone, 0.5% yeast extract and 0.5% sodium chloride was used throughout the work. Lysogeny broth agar (LBA) is similarly composed with the addition of 12 g/L agar. If required, media were supplemented with the antibiotics ampicillin (100 μg/mL), spectinomycin (100 μg/μL), kanamycin (50 μg/mL), chloramphenicol (34 μg/mL) or tetracycline (10 μg/mL).

Plasmids

Plasmid pGEM-P14-pgaABCD is made up of the origin of replication and the ampicillin resistance cassette originating from pGEM-T (Promega). The pgaABCD operon with its natural terminator was amplified from the E. coli genome to which the P14 promoter (De Mey, M. et al., BMC Biotechnol., 7) was added in the oligonucleotides.

Plasmid pGEM-T-thyA was recreated according to Stringer et al. (2012). This plasmid was constructed using CPEC according to Tian & Quan (2009). Plasmid pSC101-attPCC-P14-pgaABCD-attPTT on the other hand was made using Golden Gate according to Engler et al. (2009) in which BsaI sites were added in the primers prior to amplification. The vector backbone originated from pDonor7 while the insert sequences were derived from the respective plasmids mentioned above. Plasmid pSC101-attPCC-pYcaI-NM-msbA-attPTT was derived from pSC101-attPCC-P14-NM-msbA-attPTT in which the P14 promoter was replaced by the endogenous promoter of the ycaI-msbA operon, respectively.

Thymidylate Synthase A Selection System for the Creation of waaA Mutants

To create the waaA mutants in the thymine auxotroph base strain, the thyA cassette was amplified from pGEM-T-thyA with 50 bp homologous overhangs. The transformation protocol was followed according to Datsenko & Wanner (2000). Cultures of the auxotroph base strain were always supplemented with 30 μg/mL thymidine to allow growth. After transformation, plating was done on LBA plates without thymidine to allow selection.

SIRE Protocol

The SIRE knock-out and knock in protocol as described by Snoeck et al. (Biotechnology and Bioengineering 116 (2): 364-374) was used to create all other modifications herein.

### Example 2: Production of a Viable Escherichia coli Strain Lacking LPS

PNAG or poly-N-acetylglucosamine is a linear polymer of β-1,6-N-acetylglucosamine residues and is coded by the pgaABCD cluster in Escherichia coli. To overproduce PNAG, the pgaABCD cluster was cloned on a high copy plasmid (up to 500 copies) due to the pUC origin of replication. A strong P14 promoter was used together with the natural terminator of the cluster.

The created plasmid pGEM-P14-pgaABCD was transformed in a thymine auxotroph wild-type strain where the waaA, a normally essential gene, was targeted for deletion. Surprisingly, viable colonies were obtained showing the complete knock-out of waaA. To make sure that the overexpression of pgaABCD is the actual suppressor mutation, known suppressor mutations found in Mamat et al. (2007, Mol Microbiol. 2008 February; 67(3):633-48) such as msbA52, msbA148 and yhjD400 were checked and were not present.

1. A viable bacterial host cell with a mutated KDO₂-LipidA biosynthetic pathway leading to disruption of the pathway, wherein the host cell further comprises:
a mutation in the expression or the coding sequence of at least one gene encoding protein(s) selected from the group consisting of poly-β-1,6-N-acetyl-D-glucosamine outer membrane porin, poly-β-1,6-N-acetyl-D-glucosamine N-deacetylase, poly-N-acetyl-D-glucosamine synthase, and a combination of any thereof.

2. Host cell according to claim 1, wherein the host cell has an outer membrane lacking keto-deoxyoctulosonate (KDO).

3. Host cell according to claim 1, wherein the host cell is substantially free of lipopolysaccharide (LPS) expression.

4. Host cell according to claim 1, wherein the host cell displays LipidA in its outer membrane and/or displays LipidIVA in its outer membrane.

5. Host cell according to claim 1, wherein the disruption in the outer membrane biosynthetic pathway is caused by at least one mutation in:
a gene encoding a protein selected from the group consisting of D-arabinose 5-phosphate isomerase, KDO8P synthase, cytidine 5′-monophospho-3-deoxy-d-manno-2-octulosonic acid (CMP-KDO) synthetase, KDO8P phosphatase, and keto-deoxyoctulosonate (KDO)-transferase,
a gene or expression of a gene encoding a protein selected from the group consisting of gutQ, kdsD, kdsA, kdsB, kdsC and waaA, wherein gutQ and kdsD encode a D-arabinose 5-phosphate isomerase, kdsA encodes a KDO8P synthase, kdsB encodes a CMP-KDO synthetase, kdsC encodes a KDO8P phosphatase, and waaA encodes a KDO transferase, and/or
the gene encoding (KDO)-transferase.

6. (canceled)

7. (canceled)

8. Host cell according to claim 1, wherein the host cell is additionally mutated in:
at least one gene or the expression of at least one gene encoding LpxL and/or LpxM;
in the expression or the coding sequence of a gene selected from an adenosine triphosphate (ATP)-dependent translocator, inner membrane protein, D-arabinose 5-phosphate isomerase, Palmitoleoyl acyltransferase, Lipid A palmitoyltransferase, and/or phosphoethanolamine transferase; and/or
in the expression or the coding sequence of a gene selected from the group consisting of msbA, yhjD, gutQ, kdsD, lpxP, pagP, and eptA.

9. Host cell according to claim 1, wherein poly-β-1,6-N-acetyl-D-glucosamine outer membrane porin is encoded by pgaA, poly-β-1,6-N-acetyl-D-glucosamine N-deacetylase is encoded by pgaB, and/or poly-N-acetyl-D-glucosamine synthase activity is encoded by pgaD and pgaC.

10. Host cell according to claim 1, wherein the mutation in the expression or the coding sequence of at least one gene encoding protein(s):
poly-β-1,6-N-acetyl-D-glucosamine outer membrane porin, poly-β-1,6-N-acetyl-D-glucosamine N-deacetylase, and/or a poly-N-acetyl-D-glucosamine synthase provides poly-N-acetylglucosamine overproduction;
poly-β-1,6-N-acetyl-D-glucosamine outer membrane porin, poly-β-1,6-N-acetyl-D-glucosamine N-deacetylase, and/or poly-N-acetyl-D-glucosamine synthase so as to provide an increased activity of at least one of the proteins;
poly-β-1,6-N-acetyl-D-glucosamine outer membrane porin, poly-β-1,6-N-acetyl-D-glucosamine N-deacetylase, and/or poly-N-acetyl-D-glucosamine synthase provided by an overexpression in the pgaABCD cluster;
poly-β-1,6-N-acetyl-D-glucosamine outer membrane porin, poly-β-1,6-N-acetyl-D-glucosamine N-deacetylase, and/or poly-N-acetyl-D-glucosamine synthase provided by overexpressing or introducing and expressing or overexpressing, at least one gene encoding protein(s) a Na⁺/H⁺ antiporter regulator and a sensor histidine kinase; and/or
poly-β-1,6-N-acetyl-D-glucosamine outer membrane porin, poly-β-1,6-N-acetyl-D-glucosamine N-deacetylase, and/or poly-N-acetyl-D-glucosamine synthase provided with a reduced or abolished expression of a carbon storage regulator.

11. Host cell according to claim 1, wherein at least two of the genes encoding protein(s) poly-β-1,6-N-acetyl-D-glucosamine outer membrane porin, poly-β-1,6-N-acetyl-D-glucosamine N-deacetylase, and/or poly-N-acetyl-D-glucosamine synthase, are clustered in an operon.

12. (canceled)

13. (canceled)

14. (canceled)

15. Host cell according to claim 10, wherein the Na⁺/H⁺ antiporter regulator is a nhaR protein and/or the sensor histidine kinase is an resC protein.

16. (canceled)

17. Host cell according to claim 1, comprising a mutation that eliminates association between KDO₂ and LipidIV_{A}.

18. (canceled)

19. Host cell according to claim 5, wherein the disruption in the outer membrane biosynthetic pathway results in a lack of expression of D-arabinose 5-phosphate isomerase (API).

20. (canceled)

21. (canceled)

22. Host cell according to claim 1, wherein the host cell is a Gram-negative bacterial cell.

23. Host cell according to claim 1, wherein the host cell is selected from the group consisting of Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Mycobacteria spp., Citrobacter spp., Chlamydia spp., Brucella spp., Pseudomonas spp., Helicobacter spp. and Vibrio spp.

24. Host cell according to claim 1, wherein the host cell is further transformed with at least one gene of interest operably linked to a promoter and/or untranslated region (UTR).

25. Host cell according to claim 24, wherein the gene of interest is on a plasmid or chromosome and is expressed in the host cell.

26. Host cell according to claim 10, wherein
poly-β-1,6-N-acetyl-D-glucosamine outer membrane porin is a polypeptide having 80% or more sequence identity to SEQ ID NO:7 and having poly-β-1,6-N-acetyl-D-glucosamine transmembrane transporter activity,
poly-β-1,6-N-acetyl-D-glucosamine N-deacetylase is a polypeptide having 80% or more sequence identity to SEQ ID NO:8 and having poly-β-1,6-N-acetyl-D-glucosamine N-deacetylase activity,
poly-N-acetyl-D-glucosamine synthase is a polypeptide having 80% or more sequence identity to SEQ ID NO:9 or SEQ ID NO:10 and having poly-N-acetyl-D-glucosamine synthase activity,
ATP-dependent translocator is a polypeptide having 80% or more sequence identity to SEQ ID NO:16 and having ATP-dependent translocator activity,
inner membrane protein is a polypeptide having 80% or more sequence identity to SEQ ID NO:17 and having transmembrane transporter activity,
lauroyl acyltransferase is a polypeptide having 80% or more sequence identity to SEQ ID NO:11 and having lauroyl acyltransferase activity,
D-arabinose 5-phosphate isomerase is a polypeptide having 80% or more sequence identity to SEQ ID NO:2 or SEQ ID NO:1 and having D-arabinose 5-phosphate isomerase activity,
Palmitoleoyl acyltransferase is a polypeptide having 80% or more sequence identity to SEQ ID NO:18 and having Palmitoleoyl acyltransferase activity,
Lipid A palmitoyltransferase is a polypeptide having 80% or more sequence identity to SEQ ID NO:19 and having Lipid A palmitoyltransferase activity,
phosphoethanolamine transferase is a polypeptide having 80% or more sequence identity to the sequence as given by SEQ ID NO:20 and having phosphoethanolamine transferase activity,
myristoyl-acyl carrier protein-dependent acyltransferase is a polypeptide having 80% or more sequence identity to SEQ ID NO:12 and having myristoyl-acyl carrier protein-dependent acyltransferase activity,
KDO8P synthase is a polypeptide having 80% or more sequence identity to SEQ ID NO:3 and having KDO8P synthase activity,
CMP-KDO synthetase is a polypeptide having 80% or more sequence identity to SEQ ID NO:4 and having CMP-KDO synthetase activity,
KDO transferase is a polypeptide having 80% or more sequence identity to SEQ ID NO:6 and having KDO transferase activity,
KDO8P phosphatase is a polypeptide having 80% or more sequence identity to SEQ ID NO:5 and having KDO8P phosphatase activity,
Na⁺/H⁺ antiporter regulator is a polypeptide having 80% or more sequence identity to SEQ ID NO:13 and having Na⁺/H⁺ antiporter regulator activity,
a sensor histidine kinase is a polypeptide having 80% or more sequence identity to SEQ ID NO:14 and having sensor histidine kinase activity, and/or
a carbon storage regulator is a polypeptide having 80% or more sequence identity to SEQ ID NO:15 and having carbon storage regulator activity.

27. The host cell according to claim 1, which is isolated.

28. A composition comprising:
an outer membrane derived from the bacterial host cell of claim 1.

29. A composition comprising the host cell of claim 1.

30. (canceled)

31. (canceled)

32. (canceled)

33. A method for inducing an immune response in a subject, the method comprising:
administering a composition of claim 28 to the subject so as to induce an immune response in the subject.

34. A method of producing LipidIV_{A}, the method comprising:
extracting LipidIV_{A} from the host cell of claim 1.

35. A method for producing LipidA, the method comprising:
extracting LipidA from the host cell of claim 1.

36. A method of screening for an anti-pyrogenic agent, the method comprising:
utilizing the host cell according to claim 1 to screen for an anti-pyrogenic agent.

37. A method for producing a bioproduct or metabolite using a genetically modified host cell, the method comprising the steps of:
obtaining a host cell, which host cell has been genetically modified, such, that the host cell is able to produce the bioproduct or metabolite wherein the unmodified host cell is not able to produce the bioproduct or metabolite, due to the introduction of at least one heterologous gene, encoding the bioproduct or an intermediate thereof, which is expressed in the host cell; and
cultivating and/or growing the host cell in a cultivation medium enabling production of the bioproduct or metabolite thereby producing the bioproduct or metabolite obtainable from the medium the host cell is cultivated in;
wherein the host cell is a bacterial host cell according to claim 1.

38. A method of using the host cell of claim 1 for the production of a bioproduct or metabolite, the method comprising:
culturing the host cell
so as to produce the bioproduct or metabolite.

39. The method according to claim 38 for producing Lipid IV_{A} or Lipid A.

40. A method of immunizing a subject, the method comprising:
administering to the subject the host cell according to claim 1 to the subject.

41. A method for inducing an immune response in a subject, the method comprising:
administering the composition of claim 29 to the subject so as to induce an immune response in the subject.
